(19) United States
(12) Patent Application Publication
(10) Pub. No.: US 2021/0347849 A1
(43) Pub. Date: Nov. 11, 2021

(54) Composition for Preventing or Treating Immune-Related Diseases

(71) Applicant: Good T Cells, Inc., Seoul (KR)

(72) Inventors: Jung Ho Kim, Seoul (KR); Beom Seok Kim, Seoul (KR)

(21) Appl. No.: 17/262,629

(22) PCT Filed: Jul. 24, 2019
(86) PCT No.: PCT/KR2019/009202

§ 371 Date: Jan. 22, 2021

(30) Foreign Application Priority Data
Jul. 24, 2018 (KR) 10-2018-0085908

(51) Int. Cl. C07K 14/705; A61K 45/06

(52) U.S. Cl. CPC C07K 14/70532; A61K 38/00; A61K 45/06

(57) ABSTRACT

The present invention relates to a composition for preventing, ameliorating, or treating an immune-related disease, comprising, as an active ingredient, a fusion protein that includes an extracellular domain of leucine-rich and immunoglobulin-like domains-1 (Lrig-1) protein and an immunoglobulin Fc region. The fusion protein provided in the present invention interacts with a ligand for the Lrig-1 protein, which is present in activated T cells, to give a co-inhibitory signal to the activated T cells, so that death or suppressed activity of the activated T cells is induced, which makes it possible to effectively prevent, ameliorate, or treat an immune-related disease.

### CROSS-REFERENCE TO RELATED APPLICATIONS

This application is a U.S. National Stage entry of International Patent Application no. PCT/KR2019/009202, filed Jul. 24, 2019, which claims the benefit of priority of Korean Patent Application no. 10-2018-0085908, filed Jul. 24, 2018.

### TECHNICAL FIELD

The present invention relates to a composition capable of effectively preventing or treating immune-related diseases.

### BACKGROUND ART

Immunoglobulin comprises four polypeptide chains, that is, two heavy chains and two light chains which are associated with each other via interchain disulfide bonds. Each light chain has two domains, that is, a variable light domain (VL) and a constant light domain (CL); and each heavy chain has two regions, that is, a variable heavy region (VH) and a constant heavy region (CH). The constant heavy region (CH) consists of constant heavy regions (for example, CH1, CH2, CH3, and the like) designated by the number (see, for example, U.S. Pat. No. 6,086,875 (Blumberg R. S. et al.), U.S. Pat. No. 5,624,821 (Winter G. P. et al.), and U.S. Pat. No. 5,116,964 (Capon D. J. and Lasky L. A.)). Immunoglobulins are classified into different isotypes (that is, IgG, IgM, IgA, IgD, and IgE) based on their biological properties, location within an organism, and ability to process different antigens. Depending on the immunoglobulin isotype, the constant heavy region (CH) may have 3 or 4 CH domains. In addition, in some isotypes (IgA, IgD, and IgG), the heavy chain contains a hinge region that adds flexibility to the molecule (Janeway et al. 2001, Immunobiology, Garland Publishing, N.Y., N.Y).

In humans, there are four IgG subclasses (IgG1, 2, 3, and 4), and these IgGs are named in the order of their abundance in serum (that is, IgG1 is most abundant). The IgG isotype consists of two light chains and two heavy chains, in which each heavy chain includes three constant heavy domains (CH1, CH2, and CH3). The two heavy chains of IgG are linked to each other by disulfide bonds (—S—S—) and each heavy chain is linked to a light chain by disulfide bonds. The antigen binding site of IgG is located in the fragment antigen binding region (Fab region) that includes the variable domains of light chain (VL) and heavy chain (VH) as well as the constant domains of light chain (CL) and heavy chain (CH1). The fragment crystallizable region (Fc region) of IgG is a portion of the heavy chain containing the CH2 and CH3 domains which bind to Fc receptors found on the surface of certain cells, including a neonatal Fc receptor (FcRn). The heavy chain of IgG also has a hinge region between CH1 and CH2 which separates the Fab region from the Fc region and participates in linking the two heavy chains together via disulfide bonds. The structure of the hinge region contributes to unique biological properties of each of the four IgG subclasses.

IgG is secreted as a monomer that is small in size, allowing it to easily perfuse tissues. It is the only isotype that has a receptor (neonatal Fc receptor (FcRn)) to facilitate passage through the human placenta, thereby providing protection to the fetus in utero. IgG absorbed through the placenta provides the neonate with humoral immunity before its own immune system develops.

The IgG neonatal Fc receptor binding site is located in the Fc region of the antibody. FcRn is normally expressed in human placenta and epithelial cells and participates in an endocytic salvage pathway that prevents degradation of IgG. This salvage pathway is mediated by the highly pH-dependent binding affinity of IgG to FcRn at acidic pH. The high affinity of IgG to FcRn at acidic pH is believed to result in binding of internalized IgG to FcRn following its uptake into acidic endosomes (Goebl N A et al., 2008; Junghans R P et al., 1996). Most soluble proteins are directed to lysosomes after internalization; however, internalized FcRn-bound IgG returns to the plasma membrane and is effectively rescued from the underlying degradation pathway. Upon exposure to neutral pH in the extracellular space, IgG can dissociate from FcRn and return to the circulation. Thus, the extended serum half-life property of the antibody is retained in the Fc fragment.

The salvage pathway provides one mechanism for developing next-generation protein drugs with an extended half-life in blood circulation as compared with unmodified protein drugs. In particular, unmodified protein drugs have a short circulating half-life, and thus require frequent administration over a long-term treatment period that is needed. Extensive efforts have been made to extend the half-life of the protein drugs using a large number of approaches including PEGylation and fusion-protein technology (U.S. Food and Drug Administration; Osborn B L et al., 2002); however, these efforts have not produced ideal results.

The immune disease is a disease in which components of the mammalian immune system cause, mediate, or otherwise contribute to pathological conditions in a mammal. In particular, an inflammatory disorder is one of the most important health problems in the world. Inflammation is usually a localized protective response of body tissues against host invasion by foreign substances or harmful stimuli. Causes of inflammation include infectious agents such as bacteria, viruses, and parasites; physical factors such as burns or irradiation; chemicals such as toxins, drugs, or industrial agents; immunological responses such as allergies and autoimmune responses; or conditions associated with oxidative stress.

Inflammation is characterized by pain, redness phenomenon, swelling, heat, and eventual loss of function in an affected area. These symptoms are results of a series of complex interactions between cells in the immune system. Cellular responses result in a network of interactions between several groups of inflammatory mediators: proteins (for example, cytokine, enzyme (for example, protease, peroxidase)), major basic proteins, adhesion molecules (ICAM, VCAM), lipid mediators (for example, eicosanoid, prostaglandin, leukotriene, platelet activating factor (PAF)), reactive oxygen species (for example, hydroperoxide, superoxide anion O2-, nitric oxide (NO), and the like). However, most of these inflammatory mediators are also regulators of normal cellular activity. Thus, lack of inflammatory responses causes a host to be in an uncontrolled state so that the host is damaged (that is, infected). As a result, chronic inflammation partially leads to overproduction of several of the aforementioned mediators, by which an inflammatory disease is caused.

In addition, autoimmune disease, which is one of immune diseases, is characterized by spontaneous responses caused by the immune system attacking its own organs. These responses are due to recognition of auto-antigens by T lymphocytes, which causes a humoral immune response (production of autoantigens) and a cellular immune response (increased cytotoxic activity of lymphocytes and macrophages). Examples of the autoimmune disease may include: rheumatic diseases, psoriasis, systemic dermatomyositis, multiple sclerosis, lupus erythematosus, or deterioration of immune responses by antigens, that is, asthma, drug or food allergies, and the like. All of these diseases are restrictive and chronic diseases, which are fatal in some cases. To date, there is no effective treatment method that can treat the aforementioned diseases. Therefore, drugs, medicines, or media capable of reducing or alleviating these diseases during progression thereof will be an important solution for a patient's health.

### Technical Problem

Many researchers have been making intensive efforts to find suitable drugs and methods by searching for treatment methods for autoimmune diseases. Today, treatment of autoimmune diseases is mainly based on use of immunosuppressive drugs such as glucocorticoids, calcineurin inhibitors, and antiproliferatives-antimetabolites. However, such pharmacological therapy acts on a variety of targets, and thus may result in decreased immune function as a whole. Otherwise, in a case where this pharmacological therapy is used for a long time, various cytotoxic actions cause problems and suppress the immune system in a non-specific manner, so that a patient may be at risk for infectious diseases or immune-related diseases. Calcineurin and glucocorticoids may pose other problems due to their nephrotoxicity and diabetogenic properties, and thus their use is restricted in a case where there are some clinical symptoms (for example, renal insufficiency, diabetes, and the like).

Therefore, as a substance capable of treating immune-related diseases such as autoimmune diseases, inflammatory diseases, and transplant rejection, there is a need to develop a new therapeutic agent that exhibits an excellent therapeutic effect with no adverse effects.

### Solution to Problem

An object of the present invention is to provide a composition for preventing, ameliorating, or treating an immune-related disease, comprising, as an active ingredient, a fusion protein that includes an extracellular domain of leucine-rich and immunoglobulin-like domains-1 (Lrig-1) protein and an immunoglobulin Fc region.

Another object of the present invention is to provide a method for preventing, ameliorating, or treating the immune-related disease.

However, the technical problem to be achieved by the present invention is not limited to the above-mentioned problems, and other problems that are not mentioned will be clearly understood by those of ordinary skill in the art from the following description.

1. Composition for Preventing, Ameliorating, or Treating Immune-Related Disease

In an embodiment of the present invention, there is provided a composition for preventing, ameliorating, or treating an immune-related disease.

The composition of the present invention can be used as a pharmaceutical composition; a food composition; or a cosmetic composition.

The composition of the present invention comprises, as an active ingredient, a fusion protein that includes an extracellular domain of leucine-rich and immunoglobulin-like domains-1 (Lrig-1) protein and an immunoglobulin Fc region; a nucleic acid molecule encoding the same; an expression vector containing the nucleic acid molecule; or a host cell transformed with the expression vector.

Hereinafter, each configuration of the present invention will be described in detail.

Active Ingredient

1) Fusion Protein

Extracellular Domain of Lrig-1 Protein

In the present invention, the Lrig-1 protein is a transmembrane protein consisting of 1091 amino acids present on the surface of regulatory T immune cells, and is composed of leucine-rich repeats (LRRs) and three immunoglobulin-like domains on the extracellular or lumen side, a cell transmembrane sequence, and a cytoplasmic tail portion. The LRIG gene family includes LRIG1, LRIG2, and LRIG3, and the amino acids therebetween are highly conserved. The LRIG1 gene is highly expressed in normal skin and can be expressed in basal and hair follicle cells to regulate proliferation of epithelial stem cells.

In an example of the present invention, the extracellular domain of the Lrig-1 protein may be an extracellular domain of Lrig-1 protein derived from mammals, including primates such as humans and monkeys, rodents such as mice and rats, and the like. For the purposes of the present invention, the extracellular protein of the Lrig-1 protein may be, but is not limited to, an extracellular domain of Lrig-1 protein derived from humans or mice.

In an example of the present invention, the extracellular domain of the Lrig-1 protein may be represented by, but is not limited to, SEQ ID NO: 1 that corresponds to a sequence of amino acids 35 to 794 of the human-derived Lrig-1 protein and can be encoded by the nucleic acid sequence represented by SEQ ID NO: 2 (see Table 1).

In another example of the present invention, the extracellular domain of the Lrig-1 protein may be represented by, but is not limited to, SEQ ID NO: 3 that corresponds to a sequence of amino acids 35 to 794 of the mouse-derived Lrig-1 protein and can be encoded by the nucleic acid sequence represented by SEQ ID NO: 4 (see Table 2).

Immunoglobulin Fc Region

As used herein, the term “immunoglobulin Fc region” refers to a region of immunoglobulin which includes the heavy chain constant region 2 (CH2) and/or the heavy chain constant region 3 (CH3), excluding the heavy and light chain variable regions. The immunoglobulin Fc region may be a component that constitutes a moiety in the protein combination of the present invention.

In the present invention, the immunoglobulin Fc region may include a hinge portion in the heavy chain constant region so as to not only affect structural flexibility of the fusion protein to be finally prepared, but also further increase productivity and stability of the fusion protein. However, the present invention is not limited thereto.

In addition, the immunoglobulin Fc region of the present invention may be an extended Fc region of immunoglobulin which includes some or all of the heavy chain constant region 1 (CH1) and/or the light chain constant region 1 (CL1), excluding only the heavy and light chain variable regions, as long as the immunoglobulin Fc region has an effect that is substantially the same or improved as compared with its native type. In addition, the immunoglobulin Fc region may be a region obtained by removal of a significantly long partial amino acid sequence that corresponds to CH2 and/or CH3.

For example, the immunoglobulin Fc region of the present invention may be 1) CH1 domain, CH2 domain, CH3 domain, and CH4 domain; 2) CH1 domain and CH2 domain; 3) CH1 domain and CH3 domain; 4) CH2 domain and CH3 domain; 5) a combination of an immunoglobulin hinge region (or part of the hinge region) with one or two or more domains of CH1 domain, CH2 domain, CH3 domain, and CH4 domain and; and 6) a dimer formed of each domain of the heavy chain constant region and the light chain constant region. However, the present invention is not limited thereto.

In the present invention, the immunoglobulin Fc region of the present invention includes its native amino acid sequence as well as a sequence derivative thereof. An amino acid sequence derivative refers to an amino acid sequence that differs from its native amino acid sequence by deletion, insertion, or non-conservative or conservative substitution of at least one amino acid residue, or a combination thereof.

In the present invention, for example, for IgG Fc, the amino acid residues at positions 214 to 238, 297 to 299, 318 to 322, or 327 to 331, which are known to be important for binding, may be used as suitable sites for modification.

In the present invention, various types of derivatives are possible, including one obtained by deletion of a site capable of forming a disulfide bond, one obtained by deletion of some amino acid residues at the N-terminus of native Fc, one obtained by addition of a methionine residue at the N-terminus of native Fc, and the like. In addition, to remove an effector function therein, a complement-binding site, such as a Clq-binding site, may be deleted, or an antibody dependent cell mediated cytotoxicity (ADCC) site may be deleted. Techniques for preparing such sequence derivatives of the immunoglobulin Fc region are disclosed in International Patent Publication Nos. WO 97/34631 and WO 96/32478, and the like.

In the present invention, amino acid exchanges in proteins and peptides, which do not alter the molecular activity as a whole, are known in the art (H. Neurath, R. L. Hill, The Proteins, Academic Press, New York, 1979). The most common exchanges are exchanges occurring between the amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, or Asp/Gly. As the case may be, modification may be achieved by phosphorylation, sulfation, acrylation, glycosylation, methylation, farnesylation, acetylation, amidation, and the like.

In the present invention, the above-described Fc derivatives exhibit biological activity equivalent to that of the Fc region of the present invention, and may be those having increased structural stability against heat, pH, or the like.

In the present invention, the Fc region may be obtained from native types isolated in vivo from animals such as humans, cows, goats, pigs, mice, rabbits, hamsters, rats, or guinea pigs, or may be recombinants, which are obtained from transformed animal cells or microorganisms, or derivatives thereof. Here, the method of obtaining the Fc region from the native types may be a method in which the entire immunoglobulin is isolated from a living human or animal body, and then treated with protease to obtain the Fc region. In a case of being treated with papain, the entire immunoglobulin is cleaved into Fab and Fc; and in a case of being treated with pepsin, the entire immunoglobulin is cleaved into pF′c and F(ab)₂. Fc or pF′c may be isolated using size-exclusion chromatography or the like. In a more specific embodiment, the immunoglobulin Fc region is a recombinant immunoglobulin Fc region obtained from a microorganism using a human- or mouse-derived Fc region.

In the present invention, the immunoglobulin Fc region may have native-type glycan, or increased or decreased level of glycan as compared with the native-type, or may be in a deglycosylated form. Conventional methods such as chemical methods, enzymatic methods, and genetic engineering methods using microorganisms may be used for such increase or decrease of glycan or deglycosylation in the immunoglobulin Fc. Here, the immunoglobulin Fc region, in which glycan has been removed on Fc, exhibits remarkably decreased binding capacity with a complement (C1q) and has decreased or eliminated antibody-dependent cytotoxicity or complement-dependent cytotoxicity; and therefore does not cause any unnecessary immune responses in vivo. In this viewpoint, the deglycosylated or aglycosylated immunoglobulin Fc region would be a more suitable form for the original purpose as a drug carrier.

As used herein, the term “deglycosylation” refers to an Fc region from which sugar is enzymatically removed, and the term “aglycosylation” refers to an Fc region that is not glycosylated by being produced in a prokaryote that is E. coli in a more specific embodiment.

Meanwhile, the immunoglobulin Fc region may be derived from humans or other animals such as cows, goats, pigs, mice, rabbits, hamsters, rats, and guinea pigs. In a more specific embodiment, it is derived from humans or mouse.

In addition, the immunoglobulin Fc region of the present invention may be an IgG-, IgA-, IgD-, IgE-, or IgM-derived Fc region, heavy chain constant region 2 (CH2), heavy chain constant region 3 (CH3), hinge, a fragment thereof, or a combination thereof, or a hybrid Fc including the combination.

Meanwhile, as used herein, the term “combination” means that a polypeptide coding for single-chain immunoglobulin Fc region, heavy chain constant region 2 (CH2), or heavy chain constant region 3 (CH3), which is of the same origin, forms a bond with a single-chain polypeptide of a different origin when they form a dimer or multimer. That is, it is possible to prepare a dimer or a multimer from two or more fragments selected from IgG-, IgA-, IgM-, IgD-, or IgE-derived Fc region, heavy chain constant region 2 (CH2), or heavy chain constant region 3 (CH3).

In the present invention, the “hybrid Fc” may be derived from combinations of human IgG subclasses or combinations of human IgD and IgG. In an embodiment, the hybrid Fc may include, for example, an IgD hinge region, and a CH2 N-terminal region+IgG4 CH2 and CH3 regions. For example, a hybrid Fc form disclosed in Korean Patent No. 0897938, which is incorporated herein by reference, may be adopted and used in the same manner. In the present invention, in a case where the hybrid Fc binds to a biologically active molecule, a polypeptide, or the like, it has an effect of increasing the serum half-life of the biologically active molecule as well as an effect of increasing an expression level of the polypeptide when the polypeptide is expressed by a nucleotide sequence encoding an Fc-polypeptide fusion protein.

As an example in the present invention, the immunoglobulin Fc region may be an IgG-, IgA-, IgM-, IgD-, or IgE-derived Fc region, or may include IgG-, IgA-, IgM-, IgD-, or IgE-derived heavy chain constant region 2 (CH2) and heavy chain constant region 3 (CH3). However, the present invention is not limited thereto.

As an example in the present invention, the immunoglobulin Fc region may be an IgG- or IgM-derived Fc region which is most abundant in human blood, or may include IgG- or IgM-derived heavy chain constant region 2 (CH2) and heavy chain constant region 3 (CH3). As another example, the immunoglobulin Fc region may be an IgG-derived Fc region known to enhance the half-life of a ligand-binding protein, or may include IgG-derived heavy chain constant region 2 (CH2) and heavy chain constant region 3 (CH3). As yet another example, the immunoglobulin Fc region may be an IgG1-, IgG2-, IgG3-, or IgG4-derived Fc region, or may include the IgG1-, IgG2-, IgG3-, or IgG4-derived heavy chain constant region 2 (CH2) and heavy chain constant region 3 (CH3). As still yet another example, the immunoglobulin Fc region may include IgG1- or IgG2-derived heavy chain constant region 2 (CH2) and heavy chain constant region 3 (CH3).

As a preferred example in the present invention, the immunoglobulin Fc region may include, but is not limited to, human IgG1-derived heavy chain constant region 2 (CH2) and heavy chain constant region 3 (CH3), which are represented by SEQ ID NO: 5, or mouse IgG2-derived heavy chain constant region 2 (CH2) and heavy chain constant region 3 (CH3), which are represented by SEQ ID NO: 6 (see Table 3 below).

TABLE 3

| SEQ ID NO | Sequence information |
|---|---|
| SEQ ID NO: 5 | LFPPKPKDTLMISRTPEVTCVVVDVSHEDPEVKFNWYV |
| | DGVEVHNAKTKPREEQYNSTYRVVSVLTVLHQDWLNGK |
| | EYKCKVSNKALPAPIEKTISKAKGQPREPQVYTLPPSR |
| | DELTKNQVSLTCLVKGFYPSDIAVEWESNCiQPIHNNY |
| | KTIPPVLDSDGSTFLYSKUFVDKSPAVQQGNVFSCSVM |
| | HEALHNHYTQKSLSLSPGK |
| SEQ ID NO: 6 | IFPPKIKDVLMISLSPIVTCVVVDVSEDDPDVQISWIN |
| | NNVEVHTAQTQTEIREDYNSTLRVVSALPIQHQDWMSC |
| | GKEFKCKVNNKDLPAPIERTISKPKGSVRAPQVYVLPP |
| | PEEEMTKKQVTLTCMVTDFNIPEDIYVENVINNGKIEL |
| | NYKNIEPVLDSDGSYFMYSKIRVEKKNWVERNSYSCSV |
| | VHEGLHNFIHTTKSFSRTPGK |

As an example of the present invention, the immunoglobulin Fc region may include an IgG-, IgA-, IgM-, IgD-, IgE-, or Abatacept-derived hinge region; and as another example, the immunoglobulin Fc region may include an IgG-, IgD-, or Abatacept-derived hinge region, or may include an IgG1-, IgG2-, IgG3-, IgG4-, IgD-, or Abatacept-derived hinge region. However, the present invention is not limited thereto.

As a preferred example in the present invention, the immunoglobulin Fc region may include one or more selected from the group consisting of a human IgG1-derived hinge region represented by SEQ ID NO: 7; a mouse IgG2-derived hinge region represented by SEQ ID NO: 8; a human IgD-derived hinge region represented by SEQ ID NO: 9; and a hinge region of Abatacept represented by SEQ ID NO: 10 (see Table 4 below), thereby increasing structural flexibility of the fusion protein to be finally prepared and remarkably enhancing productivity and stability of the fusion protein. However, the present invention is not limited thereto.

TABLE 4

| SEQ ID NO | Sequence information |
|---|---|
| SEQ ID NO: 7 | EPKSSDKIFITSPPCPAPELLGGPSNIF |
| SEQ ID NO: 8 | EPRGPTIKPCPPCKCPAPNLLGGPSVF |
| SEQ ID NO: 9 | RNTGROGEEKKKEKEKEEQEERETKT |
| | PECPSHTQPLGVF |
| SEQ ID NO: 10 | EPKSSDKTHTSPPSPAPEIJLGGSSVF |

In the present invention, in a case where the extracellular domain of the Lrig-1 protein and the Fc region are connected via a linker, the linker may be linked to the N-terminus, C-terminus, or free radical of the Fc fragment and may be linked to the N-terminus, C-terminus, or free radical of the extracellular domain of the Lrig-1 protein. In a case where the linker is a peptide linker, linkage may occur at any site. For example, the linker may be linked to the C-terminus of the extracellular domain of the Lrig-1 protein and the N-terminus of the immunoglobulin Fc region, or may be linked to the C-terminus of the immunoglobulin Fc region and the N-terminus of the extracellular domain of the Lrig-1 protein.

Linker

In the present invention, the “linker” can decrease interference between the extracellular domain of the Lrig-1 protein and the immunoglobulin Fc region in the fusion protein, thereby increasing the desired activity of the extracellular domain of the Lrig-1 protein in target cells. In addition, in the present invention, the linker may include a sequence that can be cleaved by an enzyme overexpressed in the tissue or cell with the target disease. In a case where the linker can be cleaved by the enzyme overexpressed as described above, it is possible to effectively prevent the activity of the polypeptide from being decreased due to the Fc portion.

As an example of the present invention, the linker preferably has 1 to 100 amino acids; however, the present invention is not limited thereto. Any peptide, which is capable of separating the extracellular domain of the Lrig-1 protein from the immunoglobulin Fc region, may be used. Although there is no particular limitation on the amino acid sequence that constitutes the linker, it is preferable to include glycine (G) and serine (S), or to include them in a repeated or random pattern. As such an example, the linker may include at least one of a peptide linker represented by SEQ ID NO: 11 and a peptide linker represented by SEQ ID NO: 12 (see Table 5 below), or may be at least one selected from the group consisting of peptide linkers, each of which consists of an amino acid sequence represented by any one of Formulas 1 to 4:

(G)ₙ [Formula 1]

(GGGGS)ₘ [Formula 2]

(EAAAK)ₚ [Formula 3]

(XP)_{q} [Formula 4]

### In Formulas 1 to 4,

n, m, and p are each independently an integer of 1 or higher; and

q is an integer of 5 to 40;

X is any one amino acid selected from the group consisting of A (Ala), C (Cys), D (Asp), E (Glu), F (Phe), G (Gly), H (His), I (Ile), K (Lys), L (Leu), M (Met), N (Asn), O (Pyl), P (Pro), Q (Gln), R (Arg), S (Ser), T (Thr), U (Sec), V (Val), W (Trp), and Y (Tyr).

TABLE 5

| SEQ ID NO | Sequence information |
|---|---|
| SEQ ID NO: 11 | GS |
| SEQ ID NO: 12 | GGG |

In the present invention, n, m, and q in Formulas 1 to 3 may be, are not limited thereto, integers of 1 or higher, with integers of 1 to 20 being preferred.

In the present invention, X in Formula 4 may be A(Ala), but is not limited thereto.

In addition, as an example in the present invention, the linker may include at least one selected from, but not limited to, the group consisting of a peptide linker (A(EAAAK)₄ALEA(EAAAK)₄A) represented by SEQ ID NO: 96; a peptide linker (PAPAP) represented by SEQ ID NO: 97; a peptide linker (AEAAAKEAAAKA) represented by SEQ ID NO: 98; a peptide linker (IEGRMD) represented by SEQ ID NO: 99; and a peptide linker (DIEGRMD) represented by SEQ ID NO: 100.

In the present invention, in a case where the fusion protein further comprises the peptide linker, it is possible to increase stability of active substances in a cell while further enhancing productivity thereof.

In addition, as an example of the linker in the present invention, a peptide linker consisting of 33 amino acids located at positions 282 to 314, more preferably 13 amino acids located at positions 292 to 304, of human albumin, which is most abundant in the blood, may be mentioned. Such a portion is exposed in most parts to the outside as viewed in a three-dimensional structure, and thus has minimized possibility of inducing an immune response in the body. However, the present invention is not limited thereto.

In addition, in the present invention, in a case where the linker and the Fc region are separately expressed and then jointed to each other, the linker may be a crosslinking agent known in the art. The crosslinking agent may be, for example, 1,1-bis(diazoacetyl)-2-phenylethane, glutaraldehyde, N-hydroxysuccinimide esters such as 4-azidosalicylic acid, imidoestesr including disuccinimidyl esters such as 3,3′-dithiobis(succinimidylpropionate), and bifunctional maleimide such as bis-N-maleimido-1,8-octane. However, the crosslinking agent is not limited thereto.

Fusion Protein Including Lrig-1 Extracellular Domain and Immunoglobulin

As an example of the fusion protein provided in the present invention, a fusion protein, including the extracellular domain of the Lrig-1 protein represented by SEQ ID NO: 1; the hinge region represented by any one of SEQ ID NOs: 7 to 10; and the human IgG1-derived heavy chain constant region 2 (CH2) and heavy chain constant region 3 (CH3) which are represented by SEQ ID NO: 5, may be mentioned (see Table 6 below). The fusion proteins represented by SEQ ID NOs: 13 to 16 in Table 6 below may be encoded by the nucleic acid sequences represented by SEQ ID NOs: 17 to 20 in Table 7 below (see Table 7 below).

An example of the fusion protein provided by the present invention, a fusion protein, including the extracellular domain of the Lrig-1 protein represented by SEQ ID NO: 3; the hinge region represented by any one of SEQ ID NOs: 7 to 10; human IgG1-derived heavy chain constant region 2 (CH2) and heavy chain constant region 3 (CH3) which are represented by SEQ ID NO: 5, may be mentioned (see Table 8 below). The fusion proteins represented by SEQ ID NOs: 21 to 24 in Table 8 below may be encoded by nucleic acid sequences represented by SEQ ID NOs: 25 to 28 in Table 9 below (see Table 9 below).

As another example of the fusion protein provided in the present invention, a fusion protein, including the extracellular domain of the Lrig-1 protein represented by SEQ ID NO: 3; the hinge region represented by any one of SEQ ID NOs: 7 to 10; and mouse IgG2-derived heavy chain constant region 2 (CH2) and heavy chain constant region 3 (CH3) which are represented by SEQ ID NO: 6, may be mentioned (see Table 10 below). The fusion proteins represented by SEQ ID NOs: 29 to 32 in Table 10 below may be encoded by the nucleic acid sequences represented by SEQ ID NOs: 33 to 36 in Table 11 below (see Table 11 below).

As yet another example of the fusion protein provided in the present invention, a fusion protein, including the extracellular domain of the Lrig-1 protein represented by SEQ ID NO: 1; the linker represented by SEQ ID NO: 11; the hinge region represented by any one of SEQ ID NOs: 7 to 10; and the human IgG1-derived heavy chain constant region 2 (CH2) and heavy chain constant region 3 (CH3) which are represented by SEQ ID NO.: 5, may be mentioned (see Table 12 below). The fusion proteins represented by SEQ ID NOs: 37 to 40 in Table 12 below may be encoded by the nucleic acid sequences represented by SEQ ID NOs: 41 to 44 in Table 13 below (see Table 13 below).

As still yet another example of the fusion protein provided in the present invention, a fusion protein, including the extracellular domain of the Lrig-1 protein represented by SEQ ID NO. 3; the linker represented by SEQ ID NO: 11; the hinge region represented by any one of SEQ ID NOs: 7 to 10; and the human IgG1-derived heavy chain constant region 2 (CH2) and heavy chain constant region 3 (CH3) which are represented by SEQ ID NO: 5, may be mentioned (see Table 14 below). The fusion proteins represented by SEQ ID NOs: 45 to 48 in Table 14 below may be encoded by the nucleic acid sequences represented by SEQ ID NOs: 49 to 52 in Table 15 below (see Table 15 below).

As still yet another example of the fusion protein provided in the present invention, a fusion protein, including the extracellular domain of the Lrig-1 protein represented by SEQ ID NO: 3; the linker represented by SEQ ID NO: 11; the hinge region represented by any one of SEQ ID NOs: 7 to 10; and the mouse IgG2-derived heavy chain constant region 2 (CH2) and heavy chain constant region 3 (CH3) which are represented by SEQ ID NO: 6, may be mentioned (see Table 16 below). The fusion proteins represented by SEQ ID NOs: 53 to 56 in Table 16 below may be encoded by the nucleic acid sequences represented by SEQ ID NOs: 57 to 60 in Table 17 below (see Table 17 below).

As still yet another example of the fusion protein provided in the present invention, a fusion protein, including the extracellular domain of the Lrig-1 protein represented by SEQ ID NO: 1; the linker represented by SEQ ID NO: 12; the linker represented by SEQ ID NO. 11; the hinge region represented by any one of SEQ ID NOs: 7 to 10; and the human IgG1-derived heavy chain constant region 2 (CH2) and heavy chain constant region 3 (CH3) which are represented by SEQ ID NO: 5, may be mentioned (see Table 18 below). The fusion proteins represented by SEQ ID NOs: 61 to 64 in Table 18 below may be encoded by the nucleic acid sequences represented by SEQ ID NOs: 65 to 68 in Table 19 (see Table 19 below).

As still yet another example of the fusion protein provided in the present invention, a fusion protein, including the extracellular domain of the Lrig-1 protein represented by SEQ ID NO: 3; the linker represented by SEQ ID NO: 12; the linker represented by SEQ ID NO: 11; the hinge region represented by any one of SEQ ID NOs: 7 to 10; and the human IgG1-derived heavy chain constant region 2 (CH2) and heavy chain constant region 3 (CH3) which are represented by SEQ ID NO: 5, may be mentioned (see Table 20 below). The fusion proteins represented by SEQ ID NOs: 69 to 72 in Table 20 below may be encoded by the nucleic acid sequences represented by SEQ ID NOs: 73 to 76 in Table 21 below (see Table 21 below).

As still yet another example of the fusion protein provided in the present invention, a fusion protein, including the extracellular domain of the Lrig-1 protein represented by SEQ ID NO: 3; the linker represented by SEQ ID NO: 12; the linker represented by SEQ ID NO: 11; the hinge region represented by any one of SEQ ID NOs: 7 to 10; and the mouse IgG2-derived heavy chain constant region 2 (CH2) and heavy chain constant region 3 (CH3) which are represented by SEQ ID NO: 6, may be mentioned (see Table 22 below). The fusion proteins represented by SEQ ID NOs: 77 to 80 in Table 22 below may be encoded by the nucleic acid sequences represented by SEQ ID NOs: 81 to 84 in Table 23 below (see Table 23 below).

2) Nucleic Acid Molecule

The composition of the present invention comprises a nucleic acid molecule encoding the fusion protein provided in the present invention.

The nucleic acid molecule of the present invention includes any nucleic acid molecule obtained by causing the amino acid sequence of the fusion protein provided in the present invention to be converted into a polynucleotide sequence as is known to those skilled in the art. Therefore, various polynucleotide sequences can be prepared due to open reading frame (ORF), all of which are also included in the nucleic acid molecule of the present invention.

3) Expression Vector

The composition of the present invention comprises an expression vector into which the isolated nucleic acid molecule provided in the present invention is inserted.

In the present invention, the “vector” is a nucleic acid molecule that is capable of transporting another nucleic acid linked thereto. One type of vector is a “plasmid,” which refers to circular double-stranded DNA into which additional DNA segments can be ligated. Another type of vector is a phage vector. Yet another type of vector is a viral vector, in which additional DNA segments can be ligated into the viral genome. Certain vectors are capable of autonomous replication in a host cell into which they are introduced (for example, bacterial vectors are episomal mammalian vectors having a bacterial origin of replication). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form of vector.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Qμ, T-even, T2, T3, T7; plant viruses. Any expression vector known, to those skilled in the art, as an expression vector can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

To facilitate purification of the nucleic acid molecule of the present invention, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

4) Host Cell

The composition of the present invention comprises a host cell transfected with the expression vector provided in the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells. However, the host cell is not limited thereto, and any cell known to those skilled in the art which can be used as a host cell line is available.

5) Combination with V-Domain Ig Suppressor of T Cell Activation (VISTA)

In the present invention, the composition may further comprise V-domain Ig suppressor of T cell activation (VISTA) or a fragment thereof.

In the present invention, the composition may further comprise a fusion protein that includes VISTA or a fragment thereof and an immunoglobulin Fc region. Here, a linker may be further included between the VISTA or a fragment thereof and the immunoglobulin Fc region.

In the present invention, the “V-domain Ig suppressor of T cell activation (VISTA)” corresponds to an immune checkpoint protein that functions to suppress immune activity against T cells. VISTA corresponds to a surface protein that includes an extracellular domain containing an IgG-V domain and a short cytoplasmic domain not containing ITAM and ITIM. VISTA is expressed in CD11b+ myeloid dendritic cells (DCs), CD4+ and CD8+ T cells, and Foxp3+CD4+ regulatory T cells, and can function to inhibit secretion and proliferation of cytokines following T cell activation. VISTA-deficient knock-out mice exhibit decreased organ function due to chronic inflammation, and show symptoms of multiple sclerosis much faster than normal mice.

In an example of the present invention, the VISTA may be derived from mammals, including primates such as humans and monkeys, rodents such as mice and rats, and the like.

In an example of the present invention, the VISTA may consist of the amino acid sequence represented by SEQ ID NO: 85, which is human-derived VISTA, or may be encoded by the nucleotide represented by SEQ ID NO: 87. However, the present invention is not limited thereto.

In an example of the present invention, the VISTA may consist of the amino acid sequence represented by SEQ ID NO: 86, which is mouse-derived VISTA, or may be encoded by the nucleotide represented by SEQ ID NO: 88. However, the present invention is not limited thereto.

In an example of the present invention, the fragment of VISTA may be, but is not limited to, an extracellular domain of VISTA.

In an example of the present invention, the extracellular domain of VISTA may consist of, but is not limited to, the amino acid sequence represented by SEQ ID NO: 89 that corresponds to a sequence of amino acids 33 to 194 of human-derived VISTA.

In an example of the present invention, the extracellular domain of VISTA may consist of, but is not limited to, the amino acid sequence represented by SEQ ID NO: 90 that corresponds to a sequence of amino acids 33 to 191 of mouse-derived VISTA.

In an example of the present invention, the fragment of VISTA may be, but is not limited to, a fragment of the extracellular domain of VISTA.

In the present invention, the fragment of the extracellular domain of VISTA may consist of an amino acid sequence represented by any one of SEQ ID NOs: 91 to 93 that are some fragments of the extracellular domain of human-derived VISTA. However, the present invention is not limited thereto.

In the present invention, the fragment of the extracellular domain of VISTA may consist of the amino acid sequence represented by SEQ ID NO: 94 or 95 that is a fragment of the extracellular domain of mouse-derived VISTA. However, the present invention is not limited thereto.

Here, description of the immunoglobulin Fc region and the linker overlaps with that described above, and thus will be omitted to avoid excessive complexity of the specification.

In the present invention, VISTA or a fragment thereof, or a fusion protein including the same, can be easily obtained by inserting, in a host cell, an expression vector, into which a nucleic acid molecule is inserted, the nucleic acid molecule being obtained by causing the amino acid sequence provided in the present invention to be converted into a polynucleotide sequence as is known to those skilled in the art, culturing the host cell, and then performing purification using chromatography or the like.

Here, description of the nucleic acid molecule, the expression vector, and the host cell also overlaps with that described above, and thus will be omitted to avoid excessive complexity of the specification.

6) Immune-Related Diseases and Combination

Immune-Related Diseases

In the present invention, the “immune related disease” is a disease caused by excessive activation and/or proliferation of various immune cells, for example, effector T cells or cytotoxic T cells and inflammatory cells, and may include, but is not limited to, autoimmune diseases; graft versus host disease; organ transplant rejection; asthma; atopy; or acute or chronic inflammatory diseases.

In the present invention, the “autoimmune disease” may be at least one selected from, but not limited to, the group consisting of rheumatoid arthritis, type 1 diabetes, systemic scleroderma, systemic lupus erythematosus, systemic lupus erythematosus, atopic dermatitis, psoriasis, alopecia areata, asthma, Crohn's disease, Behcet's disease, Sjogren's syndrome, Guillain-Barre syndrome, chronic thyroiditis, multiple sclerosis, multiple myositis, ankylosing spondylitis, fibromyalitis, and polyarteritis nodosa.

In addition, in the present invention, the “autoimmune disease” may be caused by a process in which activated T cells infiltrate cells constituting a nerve so that inflammation or apoptosis is induced, and may be, for example, a neurodegenerative disease or a neuroinflammatory disease (see Neuromolecular Med. 2005; 7(3): 255-64).

In the present invention, the neurodegenerative disease or neuroinflammatory disease may be selected from, but not limited to, the group consisting of stroke, dementia, Alzheimer's disease, Parkinson's disease, Huntington's disease, Niemann-Pick disease, prion disease, Creutzfeldt-Jakob disease, frontal temporal dementia, Lewis dementia, amyotrophic lateral sclerosis, paraneoplastic syndrome, corticobasal degeneration, multisystematic atrophy, progressive nuclear paralysis, autoimmune disease in nervous system, spinocerebellar ataxia, inflammatory and neuropathic pain, cerebrovascular disease, spinal cord injury, and tauopathy.

In the present invention, the immune-related disease, for example, the autoimmune disease may be caused by presence of a high frequency of autoimmune T cells (for example, effector T cells or cytotoxic T cells) or remarkably increased activity thereof in patients with disease, as compared with healthy controls, which, in turn, induces an excessive immune response or destroys cells of a specific organ (see Clinical & Experimental Immunology, 180: 353-360., Immunity, Vol. 17, 1-6, Jul. 2002., Ann Rheum Dis 2004; 63(Suppl II): 96-101., Immunol Res. 2013 December; 57(0): 12-22).

In the present invention, the immune-related disease can be prevented, ameliorated, or treated by interfering with intracellular signaling pathways essential for activation and/or proliferation of T cells (for example, effector T cells or cytotoxic T cells), preferably autoreactive T cells (see Ann Rheum Dis 2004; 63(Suppl II): 96-101). Specifically, the fusion protein according to the present invention can induce a co-inhibitory signal in effector T cells, thereby inhibiting proliferation of effector T cells and regulating activation thereof. Thus, the fusion protein can be used to effectively prevent, ameliorate, or treat various immune-related diseases that may be caused by excessive activation and/or proliferation of T cells (for example, effector T cells or cytotoxic T cells).

Combination with Therapeutic Agents for Immune-Related Diseases

The composition of the present invention may be used in combination with other drugs for treating the immune-related diseases. For example, the autoimmune diseases can be treated with molecules according to at least some embodiments of the present invention, together with (but not limited to) immunosuppressants such as corticosteroid, cyclosporine, cyclophosphamide, prednisone, azathioprine, methotrexate, rapamycin, and tacrolimus, biological agents such as TNF-α blocker or antagonist, or any other biological agents that target any inflammatory cytokine, nonsteroidal anti-inflammatory drug/Cox-2 inhibitor, hydroxychloroquine, sulphasalazopryine, gold salt, etanercept, infliximab, mycophenolate mofetil, basiliximab, atacicept, rituximab, cytoxan, interferon β-1a, interferon β-1b, glatiramer acetate, mitoxantrone hydrochloride, anakinra, and/or other biological substances and/or intravenous immunoglobulin (IVIG). Non-limiting examples of such known therapeutic agents include interferons such as IFN-β-1a (REBIF®, AVONEX®, and CINNOVEX® and IFN-β-1b (BETASERON®, EXTAVIA®, BETAFERON®, and ZIFERON®); glatiramer acetate (COPAXONE®) which is a polypeptide; natalizumab (TYSABRI®); and mitoxantrone (NOVANTRONE®) which is a cytotoxic agent.

In addition, the composition of the present invention may be used in combination with any known therapeutic agent or method for treating multiple sclerosis. Non-limiting examples of such a known therapeutic agent or method for treating multiple sclerosis include interferons such as IFN-β-1a (REBIF®, AVONEX®, and CINNOVEX®) and IFN-β-1b (BETASERON®, EXTAVIA®, BETAFERON®, and ZIFERON®); glatiramer acetate (COPAXONE®) which is a polypeptide; natalizumab (TYSABRI®); and mitoxantrone (NOVANTRONE®) which is a cytotoxic agent, fampridine (AMPYRA®). Other drugs include corticosteroid, methotrexate, cyclophosphamide, azathioprine and intravenous immunoglobulin (IVIG), inosine, ocrelizumab (R1594), Mylinax (Caldribine), alemtuzumab (Campath), daclizumab (Zenapax), Panaclar/dimethyl fumarate (BG-12), Teriflunomide (HMR1726), Fingolimod (FTY720), Laquinimod (ABR216062), as well as hematopoietic stem cell transplantation, Neurovx, rituximab (Rituxan), BCG vaccine, low-dose naltrexone, anthelmintic therapy, angioplasty, venous stent, and alternative therapies such as vitamin D, polyunsaturated fat, and medical marijuana.

In addition, the composition of the present invention may be used in combination with any known therapeutic agent or method for treating rheumatoid arthritis. Non-limiting examples of such a known therapeutic agent or method for treating rheumatoid arthritis include glucocorticoids, nonsteroidal anti-inflammatory drug (NSAID) such as salicylates, or cyclooxygenase-2 inhibitors, ibuprofen and naproxen, diclofenac, indomethacin, etodolac; disease-modifying antirheumatic drugs (DMARDs)-oral DMARDs: Auranofin (Ridaura®), Azathioprine (Imuran®), cyclosporine (Sandimmune®, GengrafM, and Neoral®, each of which is generic), D-penicillamine (Cuprimine®), hydroxychloroquine (Plaquenil®), IM gold sodium thiomalate (Myochrysine®), aurothioglucose (Solganal®), leflunomide (Arava®), methotrexate (Rheumatrex®), minocycline (Minocin®), staphylococcal protein A immunoadsorption (Prosorba® column), sulfasalazine (Azulfidine®); biological DMARDs: TNF-α blockers including adalimumab (Humira®), etanercept (Enbrel®), infliximab (Remicade®), golimumab (Simponi®), certolizumab pegol (Cimzia®), and other biological DMARDs, such as anakinra (Kineret®), rituximab (Rituxan®), tocilizumab (Actemra®), CD28 inhibitors including Abatacept (Orencia®) and belatacept.

In addition, the composition of the present invention may be used in combination with any known therapeutic agent or method for type 1 diabetes. Non-limiting examples of such a known therapeutic agent or method for treating type 1 diabetes include insulin, insulin analogs, islet transplantation, stem cell therapy including PROCHYMAL®, non-insulin therapies such as IL-1β inhibitors including anakinra (Kineret®), Abatacept (Orencia®), diamyd, alefacept (Ameviv®), otelixizumab, DiaPep277 (Hsp60 derived peptide), alpha 1-antitrypsin, prednisone, azathioprine, ciclosporin, E1-INT (an injectable islet neogenesis therapy comprising an epidermal growth factor analog and a gastrin analog), statins including Zocor®, Simlup®, Simcard®, Simvacor®, Sitagliptin® (dipeptidyl peptidase (DPP-4) inhibitor), anti-CD3 mAb (for example, teplizumab); CTLA4-Ig (Abatacept), anti IL-1β (canakinumab), anti-CD20 mAb (for example, rituximab).

In addition, the composition of the present invention may be used in combination with any known therapeutic agent or method for treating Sjogren's syndrome. Non-limiting examples of such a known therapeutic agent or method for treating Sjogren's syndrome include cyclosporine, pilocarpine (Salagen®) and cevimeline (Evoxac®), hydroxychloroquine (Plaquenil®), cortisone (prednisone and others) and/or azathioprine (Imuran®) or cyclophosphamide (Cytoxan®), dexamethasone, thalidomide, dehydroepiandrosterone, NGX267, rebamipide, FID 114657, Etanercept®, Raptiva®, belimumab, MabThera® (rituximab); anakinra, intravenous immunoglobulin (IVIG), allogeneic mesenchymal stem cells (AlloMSCs), automatic neuro-electrostimulation by “Saliwell Crown”.

In addition, the composition of the present invention may be used in combination with any known therapeutic agent or method for treating systemic lupus erythematosus. Non-limiting examples of such a known therapeutic agent or method for treating systemic lupus erythematosus include corticosteroids and disease-modifying antirheumatic drugs (DMARDs), commonly anti-malarial drugs such as plaquenil and immunosuppressants (for example, methotrexate and azathioprine) hydroxychloroquine, cytotoxic drugs (for example, cyclophosphamide and mycophenolate), hydroxychloroquine (HCQ), Benlysta® (belimumab), nonsteroidal anti-inflammatory drugs, prednisone, cellcept, prograf, atacicept, lupuzor, intravenous immunoglobulin (IVIG), CellCept® (mycophenolate mofetil), Orencia®, CTLA4-IgG4m (RG2077), rituximab, ocrelizumab, epratuzumab, CNTO 136, Sifalimumab (MEDI-545), A-623 (formerly AMG 623), AMG 557, rontalizumab, paquinimod (ABR-215757), LY2127399, CEP-33457, dehydroepiandrosterone, levothyroxine, abetimus sodium (LJP 394), memantine, opiates, rapamycin, renal transplantation, and stem cell transplantation.

7) Terms and Composition

In the present invention, the “prevention” may include, without limitation, any act of blocking symptoms of a disease, or suppressing or delaying the symptoms, using the composition of the present invention.

In the present invention, the “amelioration” or “treatment” may include, without limitation, any act of ameliorating or beneficially altering symptoms of a disease, using the composition of the present invention.

In the present invention, the pharmaceutical composition may be characterized by being in the form of capsules, tablets, granules, injections, ointments, powders, or beverages, and the pharmaceutical composition may be characterized by being targeted to humans.

In the present invention, the pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, excipients, or diluents suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

In the present invention, the route of administration of the pharmaceutical composition includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary widely depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way. The pharmaceutical composition according to the present invention may be formulated in the form of pills, sugar-coated tablets, capsules, liquids, gels, syrups, slurries, or suspensions.

In the present invention, the food composition may be prepared in the form of various foods, for example, beverages, gums, tea, vitamin complexes, powders, granules, tablets, capsules, confections, rice cakes, bread, and the like.

In the present invention, in a case where the active ingredient is contained in the food composition, the amount thereof to be added may be, but is not limited to, 0.1% to 50% of a total weight of the food composition.

In the present invention, in a case where the food composition is prepared in the form of a beverage, there is no particular limitation except that the beverage contains the food composition at an indicated proportion, and the beverage may contain various flavoring agents or natural carbohydrates, or the like as additional ingredients similarly to conventional beverages. Specifically, examples of the natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like).

In the present invention, the food composition may further contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like.

In the present invention, the above-mentioned ingredients may be used individually or in combination. The proportion of such additives does not correspond to a key element of the present invention, and may be selected from, but is not limited to, the range of 0.1 to about 50 parts by weight per 100 parts by weight of the food composition of the present invention.

In the present invention, the cosmetic composition may be prepared in the form of skin softeners, nourishing lotions, nourishing essences, massage creams, cosmetic bath water additives, body lotions, body milks, bath oil, baby oil, baby powders, shower gels, shower creams, sun screen lotions, sun screen creams, suntan creams, skin lotions, skin creams, UV blocking cosmetics, cleansing milks, hair removing agents (for cosmetic purposes), face and body lotions, face and body creams, skin whitening creams, hand lotions, hair lotions, cosmetic creams, Jasmine oil, bath soaps, liquid soaps, cosmetic soaps, shampoos, hand cleaners, medicinal soaps (for non-medical purposes), cream soaps, facial washes, body cleansers, scalp cleansers, hair rinses, toilet soaps, tooth whitening gels, toothpastes, and the like. The composition of the present invention may further contain either a solvent which is conventionally used for the preparation of cosmetic compositions, or a suitable carrier, excipient, or diluent.

In the present invention, the type of solvent that may further be added to the cosmetic composition is not particularly limited, and examples of the solvent may include water, saline, DMSO, or a combination thereof. In addition, examples of the carrier, excipient, or diluent include, but are not limited to, purified water, oil, wax, fatty acids, fatty acid alcohol, fatty acid esters, surfactants, humectants, thickeners, antioxidants, viscosity stabilizers, chelating agents, buffers, lower alcohol, and the like. In addition, the cosmetic composition of the present invention may, if necessary, contain whitening agents, moisturizing agents, vitamins, UV blocking agents, fragrances, dyes, antibiotics, antibacterial agents, and antifungal agents.

In the present invention, examples of the oil may include hydrogenated vegetable oil, castor oil, cottonseed oil, olive oil, palm kernel oil, jojoba oil, and avocado oil, and examples of the wax may include beeswax, spermaceti, carnauba wax, candelilla wax, montan wax, ceresin wax, liquid paraffin, and lanolin.

In the present invention, examples of the fatty acids may include stearic acid, linoleic acid, linolenic acid, and oleic acid; examples of the fatty acid alcohol may include cetyl alcohol, octyl dodecanol, oleyl alcohol, panthenol, lanolin alcohol, stearyl alcohol, and hexadecanol; and examples of the fatty acid esters may include isopropyl myristate, isopropyl palmitate, and butyl stearate. Examples of the surfactants may include cationic surfactants, anionic surfactants, and nonionic surfactants, which are known in the art. Among these, if possible, surfactants derived from natural products are preferred. In addition, the cosmetic composition of the present invention may contain humectants, thickeners, antioxidants, and the like, which are widely known in the cosmetic field, and the types and amounts thereof are as known in the art.

2. Method for Preventing, Ameliorating, or Treating Immune-Related Diseases

According to another embodiment of the present invention, there is provided a method for preventing, ameliorating, or treating an immune-related disease, comprising a step of administering, to an individual, the fusion protein according to the present invention; a nucleic acid molecule encoding the same; an expression vector containing the nucleic acid molecule; or a host cell transformed with the expression vector; or a composition including any one of the foregoing.

In the present invention, descriptions related to the fusion protein, the nucleic acid molecule, the expression vector, the host cell, the composition, and the immune-related diseases overlap with those described above for the pharmaceutical composition, and thus will be omitted to avoid excessive complexity of the specification.

In the present invention, the “individual” is an individual suspected of developing an immune-related disease, and the individual suspected of developing an immune-related disease means a mammal, such as humans, mice, and domestic animals, who has developed or is likely to develop the disease in question. However, any individual, who is treatable with the fusion protein of the present invention or the composition comprising the same, is included therein without limitation.

The method of the present invention may comprise administering a fusion protein or a composition comprising the same in a pharmaceutically effective amount. An appropriate total daily amount used may be determined by an attending physician or veterinarian within the scope of sound medical judgment, and administration may be made once or several times. However, for the purposes of the present invention, a specific therapeutically effective amount for a particular patient is preferably applied differently depending on various factors, including type and degree of reaction to be achieved, the specific composition including whether other agents are used therewith as the case may be, the patient's age, body weight, general health status, sex, and diet, time of administration, route of administration, secretion rate of the composition, duration of treatment, and drugs used simultaneously or in combination with the specific composition, and similar factors well known in the medical field.

Meanwhile, the method for preventing, ameliorating, or treating immune-related diseases may be, but is not limited to, a combination therapy that further comprises administering a compound or substance having therapeutic activity against one or more immune-related diseases.

In the present invention, the “combination” should be understood to represent simultaneous, individual, or sequential administration. In a case where the administration is made in a sequential or individual manner, the second component should be administered at intervals such that beneficial effects of the combination are not lost.

In the present invention, the dosage of the fusion protein may be, but is not limited to, about 0.0001 μg to 500 mg per kg of patient's body weight.

### Advantageous Effects of Invention

The fusion protein provided in the present invention interacts with a ligand for the Lrig-1 protein, which is present in activated T cells, to give a co-inhibitory signal to the activated T cells, so that death or suppressed activity of the activated T cells is induced, which makes it possible to effectively prevent, ameliorate, or treat immune-related diseases.

### BRIEF DESCRIPTION OF DRAWINGS

FIG. 1 illustrates a structure of the Lrig-1 protein according to an embodiment of the present invention.

FIG. 2 illustrates a structure of the Lrig-1 protein according to an embodiment of the present invention.

FIG. 3 illustrates an expression level of Lrig-1 protein according to an embodiment of the present invention.

FIG. 4 illustrates an expression level of Lrig-1 mRNA according to an embodiment of the present invention.

FIG. 5 illustrates an expression level of Lrig-1 mRNA according to an embodiment of the present invention.

FIG. 6 graphically illustrates results obtained by comparing expression levels of Lrig-1, Lrig-2, and Lrig-3 mRNAs according to an embodiment of the present invention.

FIG. 7 illustrates results obtained by comparing expression levels of Lrig-1 protein in regulatory T cells and non-regulated T cells according to an embodiment of the present invention.

FIG. 8 illustrates results obtained by identifying expression levels of Lrig-1 protein on the surface of various immune cells according to an embodiment of the present invention.

FIG. 9 illustrates subsets of T cells in which a ligand recognized by the fusion protein according to an embodiment of the present invention is present.

FIG. 10 illustrates results obtained by subjecting naive T cells, and activated T cells obtained by activation with an anti-CD3/28 antibody to staining with the fusion protein and PD-L1-IgG at respective concentrations, according to an embodiment of the present invention.

FIG. 11 illustrates results obtained by subjecting activated T cells (Act) and Th1 cells (TH1) to immunoprecipitation, and then to Western blot analysis with an anti-VISTA antibody, according to an embodiment of the present invention.

FIGS. 12A and 12B illustrate results (A) obtained by activating T cells with an anti-CD3/28 antibody, allowing a VISTA antibody to be bound to the cell surface for 30 minutes, subjecting the cell surface to staining with Lrig-1-IgG, and then performing analysis through a flow cytometer, and results (B) obtained by allowing the fusion protein to be bound to the surface of the activated T cells for 30 minutes, subjecting the cell surface to staining with a VISTA antibody, and then performing analysis through a flow cytometer, respectively, according to an embodiment of the present invention.

FIGS. 13A and 13B illustrate results (A) obtained by activating naïve T cells with an anti-CD3/28 antibody, subjecting the surface of the T cells to staining with a VISTA antibody every 24 hours, and then measuring changes in expression level of VISTA through a flow cytometer, and results (B) obtained by collecting a culture of the activated T cells every 24 hours, and then measuring, with a VISTA ELISA kit, an expression level of an extracellular domain of VISTA so that an amount of the extracellular domain present in the culture is measured.

FIGS. 14A and 14B graphically illustrate results obtained by collecting blood from normal mice and a mouse model for which rheumatoid arthritis (RA) or inflammatory bowel disease (IBD) has been induced, separating serum therefrom through centrifugation, and then measuring a concentration of an extracellular domain of VISTA in the serum using ELISA that targets the extracellular domain of VISTA, according to an embodiment of the present invention.

FIG. 15 illustrates results obtained by activating naive T cells, and then analyzing, with FACS, an expression level of CD25, which is a surface protein induced on the cell surface, according to an embodiment of the present invention.

FIGS. 16A, 16B, 16C, and 17 illustrate results obtained by identifying a proliferation inhibitory effect of the fusion protein according to the present invention in activated T cells, according to an embodiment of the present invention.

### DETAILED DESCRIPTION OF INVENTION

According to an embodiment of the present invention, there is provided a composition for preventing, ameliorating, or treating an immune-related disease, comprising, as an active ingredient, a fusion protein consisting of the amino acid sequence represented by SEQ ID NO: 21; a nucleic acid molecule encoding the same; an expression vector containing the nucleic acid molecule; or a host cell transformed with the expression vector.

According to another embodiment of the present invention, there is provided a composition for preventing, ameliorating, or treating an immune-related disease, comprising, as an active ingredient, a fusion protein encoded by the nucleotide sequence represented by SEQ ID NO: 25; a nucleic acid molecule encoding the same; an expression vector containing the nucleic acid molecule; or a host cell transformed with the expression vector.

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

### [Preparation Example 1] T Cell Subtype Cell Culture

To identify whether the Lrig-1 protein is expressed only in regulatory T cells (Treg), the subsets of T cells, Th0, Th1, Th2, Th17, and iTreg, were prepared. The iTreg refers to cells whose differentiation has been artificially induced in a medium having the following composition, unlike nTreg which has been naturally isolated.

The subsets of the T cells were induced to differentiate into respective cells by first isolating naive T cells obtained from the spleen of mice, causing RPMI1640 (Invitrogen Gibco, Grand Island, N.Y.) nutrient medium that contains 10% fetal bovine serum (FBS; HyClone, Logan, Utah) to further contain the respective ingredients of Table 24 below, and performing 72-hour incubation in an incubator at 37° C., 5% CO₂.

TABLE 24

| Differentiated cell | Composition |
|---|---|
| Th0 | anti-CD3, anti-CD28 |
| Th1 | IL-12, anti-IL-4 antibody |
| Th2 | IL-4, anti-IFNβ |
| Th17 | IL-6, TGFβ, anti-IFNβ, anti-IL-4 |
| iTreg | IL-2, TGFβ |

### [Example 1] Structural Analysis of Lrig-1

A three-dimensional steric structure of the extracellular domain of the Lrig-1 protein was predicted to produce a fusion protein including the extracellular domain of Lrig-1 protein, a surface protein of regulatory T cells.

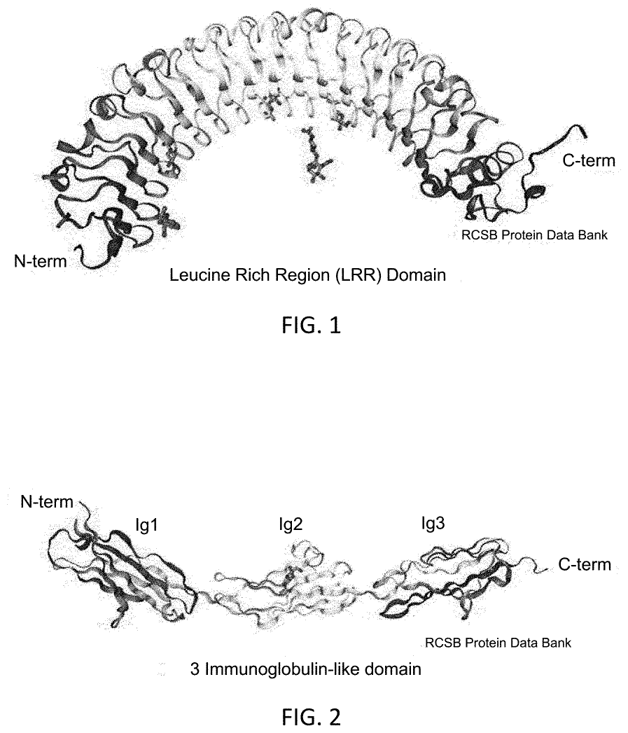

First, to predict base sequences of epitopes, tools of Uniprot (http://www.uniprot.org) and RCSB Protein Data Bank (http://www.rcsb.org/pdb) were used to predict a three-dimensional steric structure of the extracellular domain (ECD) of the Lrig-1 protein so that the structure of ECD is identified. Then, the results are illustrated in FIGS. 1 and 2.

As illustrated in FIG. 1, a total of 15 leucine-rich regions of LRR1 to LRR15 exist in the Lrig-LRR domain (amino acid sequence at positions 41 to 494) in the extracellular domain of the Lrig-1 protein. Each of the LRR domains is composed of 23 to 27 amino acids, with 3 to 5 leucine being present.

In addition, as illustrated in FIG. 2, three immunoglobulin-like domains exist in amino acid sequences at positions 494 to 781 of the Lrig-1 protein in the extracellular domain of the Lrig-1 protein.

### [Example 2] Identification of Specific Expression of Lrig-1 mRNA in Regulatory T Cells

Verification was made of whether the Lrig-1 protein can act as a biomarker specific for regulatory T cells.

For the verification, CD4⁺ T cells were isolated using magnet-activated cell sorting (MACS), through CD4 beads, from the spleen of mice. Subsequently, regulatory T (CD4⁺CD25⁺ T) cells and non-regulatory T (CD4⁺CD25⁻ T) cells were isolated with a fluorescence-activated cell sorter (FACS) using a CD25 antibody. For the respective cells and the cells differentiated in Preparation Example 1, mRNA was extracted using Trizol, and then gDNA was removed from genomic RNA using gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively identify an expression level of Lrig-1 mRNA in the cDNA.

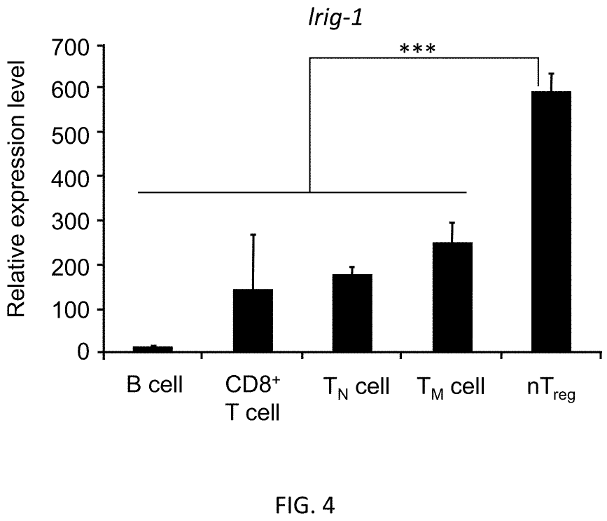

The real-time polymerase chain reaction was performed with primers shown in Table 25 below using SYBR Green (Molecular Probes) according to the protocol provided by the manufacturer under conditions of 40 cycles consisting of 95° C. for 3 minutes, 61° C. for 15 seconds, 72° C. for 30 seconds, a relative gene expression level was calculated using the ΔCT method, and normalized using HPRT. The results are illustrated in FIGS. 4 to 6.

TABLE 25

| Primer | Sequence |
|---|---|
| Mouse Lrig-1 | Forward 5′-GAC GGA ATT CAG TGA GGA |
| | GAA CCT-3′ |
| | Reverse 5′-CAA CTG GTA GTG GCA GCT |
| | TGT AGG-3′ |
| Mouse Lrig-2 | forward 5′-TCA CAA GGA ACA TTG TCT |
| | GAA CCA-3′ |
| | reverse 5′-GCC TGA TCT AAC ACA TCC |
| | TCC TCA-3′ |
| Mouse Lrig-3 | forward 5′-CAG CAC CTT GAG CTG AAC |
| | AGA AAC-3′ |
| | reverse 5′-CCA GCC TTT GGT AAT CTC |
| | GGT TAG-3′ |
| Mouse FOXP3 | forward 5′-CTT TCA CCT ATC CCA CCC |
| | TTA TCC-3′ |
| | reverse 5′-ATT CAT CTA CGG TCC ACA |
| | CTG CTC-3′ |
| ACTG1 | forward 5′-GGC GTC ATG GTG GGC ATG |
| | GG-3′ |
| | reverse 5′-ATG GCG TGG GGA AGG GCG |
| | TA-3′ |

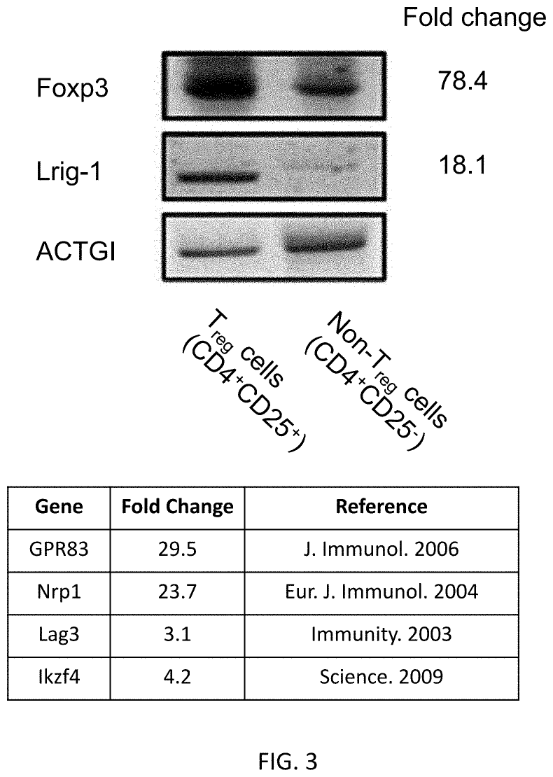

As illustrated in FIG. 3, it can be seen that the expression of Lrig-1 in regulatory T (CD4⁺CD25⁺ T) cells is 18.1 times higher than non-regulatory T (CD4⁺CD25⁻ T) cells. This was about 10 times higher expression level than Lag3 and Ikzf4, which are previously known markers for regulatory T cells.

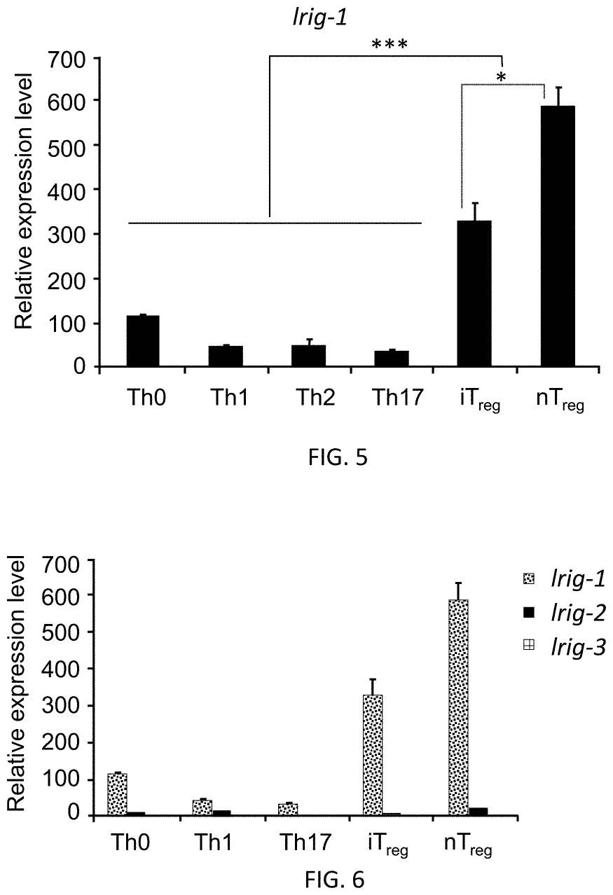

In addition, as illustrated in FIGS. 4 and 5, the expression of Lrig-1 mRNA was remarkably high in regulatory T cells as compared with other types of immune cells, and in particular, was remarkably high in naturally isolated regulatory T cells (nTreg) as compared with induced regulatory T cells (iTreg).

In addition, as illustrated in FIG. 6, the expression of Lrig-1 was the highest among Lrig-1, Lrig-2, and Lrig-3 which correspond to the Lrig family.

From the above results, it can be seen that the Lrig-1 protein according to the present invention is specifically expressed in regulatory T cells, in particular, naturally-occurring regulatory T cells.

### [Example 3] Identification of Specific Expression of Lrig-1 Protein in Regulatory T Cells

It was identified whether the Lrig-1 protein expressed from Lrig-1 mRNA is specifically expressed only in regulatory T cells.

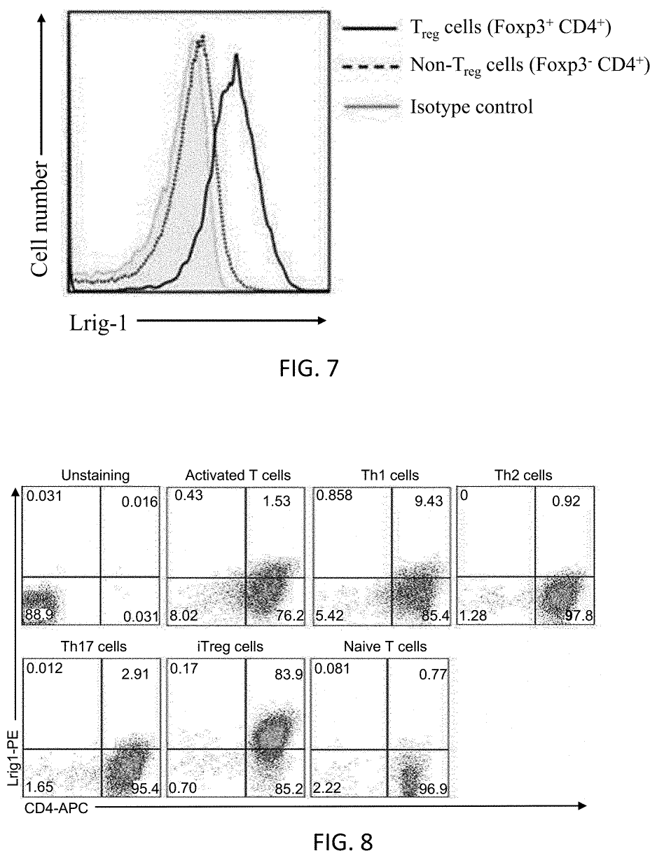

Using FOXP3-RFP-knocked-in mice, the FOXP3-RFP obtained by coupling red fluorescence protein (RFP) to FOXP3 promoter, which is a transcription factor specific for regulatory T cells, CD4⁺ T cells were isolated using magnet-activated cell sorting (MACS), through CD4 beads, from the spleen of the mice. Subsequently, using RFP protein, regulatory T (CD4⁺RFP⁺ T) cells and non-regulatory T (CD4⁺RFP⁻ T) cells were obtained by performing isolation through a fluorescence-activated cell sorter (FACS). The respective cells were stained with the purchased Lrig-1 antibody and a negative control was stained with an isotype-matched control antibody, to measure an expression level of Lrig-1 with the fluorescence-activated cell sorter. The results are illustrated in FIG. 7.

As illustrated in FIG. 7, the non-regulatory T cells indicated by a dotted line showed almost the same expression level of Lrig-1 as the negative control, whereas there were a large number of cells with high expression level of Lrig-1 in the regulatory T cells.

From the above results, it can be seen that the Lrig-1 protein according to the present invention is specifically expressed in regulatory T cells.

### [Example 4] Identification of Specific Expression of Lrig-1 Protein on Surface of Regulatory T Cells

From the viewpoint that in order to be a target of cell therapy, the Lrig-1 protein must be expressed on the surface of regulatory T cells, it was identified whether the Lrig-1 protein is expressed on the surface of the regulatory T cells.

The respective differentiated T cell subsets of Preparation Example 1 were stained with anti-CD4-APC and anti-Lrig-1-PE antibodies, and expression levels of Lrig-1 protein were measured at the respective cell surfaces using a fluorescence-activated cell sorter (FACS). The results are illustrated in FIG. 8.

As illustrated in FIG. 8, Lrig-1 was expressed in an amount of 0.77 to 15.3 in activated T cells, Th1 cells, Th2 cells, Th17 cells, and naive T cells, whereas Lrig-1 was expressed as high as 83.9 in differentiation-induced T cells (iTreg cells).

From the above results, it can be seen that the Lrig-1 protein according to the present invention is not only specifically expressed in regulatory T (Treg) cells, but also is, in particular, specifically expressed at a higher level on the surface of the Treg cells.

### [Preparation Example] Production of Fusion Protein

1. Construction of Expression Vector

To produce the fusion protein according to the present invention, respective nucleic acid sequences encoding respective fusion proteins as shown in Table 26 were synthesized. NheI and EcoRI restriction enzyme sequences were added to the 5′ and 3′ ends of the nucleic acid sequence, respectively, and the following sequences were inserted after the restriction enzyme sequence at the 5′ end: the Kozak's sequence (GCCACC), a start codon for protein translation, and the mouse IgG kappa light chain signal peptide (ATGGAAACCGATACTCTGCTGCTGTGGGTGCTGCTGCTGTGGGTGCCAGGCTCTACC GGG) that allows an expressed protein to secrete outside the cell. Subsequently, a stop codon was inserted after the nucleic acid sequence encoding each of the fusion proteins as shown in Table 26, in which the each fusion protein includes the extracellular domain of human-derived Lrig-1 protein or VISTA protein; optionally a linker; a hinge region; human IgG1-derived heavy chain constant region 2 (CH2) and heavy chain constant region 3 (CH3). The nucleic acid sequence encoding the fusion protein of the present invention was cloned into pcDNA3.1(+) expression vector using the two restriction enzyme sequences, NheI and EcoRI.

TABLE 26

| | Amino acid sequence | Nucleic acid sequence |
|---|---|---|
| No. | of fusion protein | of fusion protein |
| Preparation | SEQ ID NO: 13 | SEQ ID NO: 17 |
| Example 1 | | |
| Preparation | SEQ ID NO: 14 | SEQ ID NO: 18 |
| Example 2 | | |
| Preparation | SEQ ID NO: 15 | SEQ ID NO: 19 |
| Example 3 | | |
| Preparation | SEQ ID NO: 16 | SEQ ID NO: 20 |
| Example 4 | | |
| Preparation | SEQ ID NO: 21 | SEQ ID NO: 25 |
| Example 5 | | |
| Preparation | SEQ ID NO: 22 | SEQ ID NO: 26 |
| Example 6 | | |
| Preparation | SEQ ID NO: 23 | SEQ ID NO: 27 |
| Example 7 | | |
| Preparation | SEQ ID NO: 24 | SEQ ID NO: 28 |
| Example 8 | | |
| Preparation | SEQ ID NO: 29 | SEQ ID NO: 33 |
| Example 9 | | |
| Preparation | SEQ ID NO: 30 | SEQ ID NO: 34 |
| Example 10 | | |
| Preparation | SEQ ID NO: 31 | SEQ ID NO: 35 |
| Example 11 | | |
| Preparation | SEQ ID NO: 32 | SEQ ID NO: 36 |
| Example 12 | | |
| Preparation | SEQ ID NO: 37 | SEQ ID NO: 41 |
| Example 13 | | |
| Preparation | SEQ ID NO: 38 | SEQ ID NO: 42 |
| Example 14 | | |
| Preparation | SEQ ID NO: 39 | SEQ ID NO: 43 |
| Example 15 | | |
| Preparation | SEQ ID NO: 40 | SEQ ID NO: 44 |
| Example 16 | | |
| Preparation | SEQ ID NO: 45 | SEQ ID NO: 49 |
| Example 17 | | |
| Preparation | SEQ ID NO: 46 | SEQ ID NO: 50 |
| Example 18 | | |
| Preparation | SEQ ID NO: 47 | SEQ ID NO: 51 |
| Example 19 | | |
| Preparation | SEQ ID NO: 48 | SEQ ID NO: 52 |
| Example 20 | | |
| Preparation | SEQ ID NO: 53 | SEQ ID NO: 57 |
| Example 21 | | |
| Preparation | SEQ ID NO: 54 | SEQ ID NO: 58 |
| Example 22 | | |
| Preparation | SEQ ID NO: 55 | SEQ ID NO: 59 |
| Example 23 | | |
| Preparation | SEQ ID NO: 56 | SEQ ID NO: 60 |
| Example 24 | | |
| Preparation | SEQ ID NO: 61 | SEQ ID NO: 65 |
| Example 25 | | |
| Preparation | SEQ ID NO: 62 | SEQ ID NO: 66 |
| Example 26 | | |
| Preparation | SEQ ID NO: 63 | SEQ ID NO: 67 |
| Example 27 | | |
| Preparation | SEQ ID NO: 64 | SEQ ID NO: 68 |
| Example 28 | | |
| Preparation | SEQ ID NO: 69 | SEQ ID NO: 73 |
| Example 29 | | |
| Preparation | SEQ ID NO: 70 | SEQ ID NO: 74 |
| Example 30 | | |
| Preparation | SEQ ID NO: 71 | SEQ ID NO: 75 |
| Example 31 | | |
| Preparation | SEQ ID NO: 72 | SEQ ID NO: 76 |
| Example 32 | | |
| Preparation | SEQ ID NO: 77 | SEQ ID NO: 81 |
| Example 33 | | |
| Preparation | SEQ ID NO: 78 | SEQ ID NO: 82 |
| Example 34 | | |
| Preparation | SEQ ID NO: 79 | SEQ ID NO: 83 |
| Example 35 | | |
| Preparation | SEQ ID NO: 80 | SEQ ID NO: 84 |
| Example 36 | | |

2. Purification of Fusion Protein

293F cells were transformed with the expression vector as constructed in item no. 1. above using polyethyleneimine, and then cultured for 6 days under conditions of 37° C. and 8% CO₂. The culture supernatant was filtered, passed through Protein A resin, and washed with 1×PBS. Elution was performed with a 0.1 M glycine solution at pH 3.5. Then, to the obtained solution was added a 1 M TRIS solution at pH 9.0 to neutralize the pH. Subsequently, the solution was dialyzed against a PBS solution, and then concentrated and used.

### [Example 5] Identification of Distribution of Lrig-1 Ligand Recognized by Fusion Protein According to Present Invention

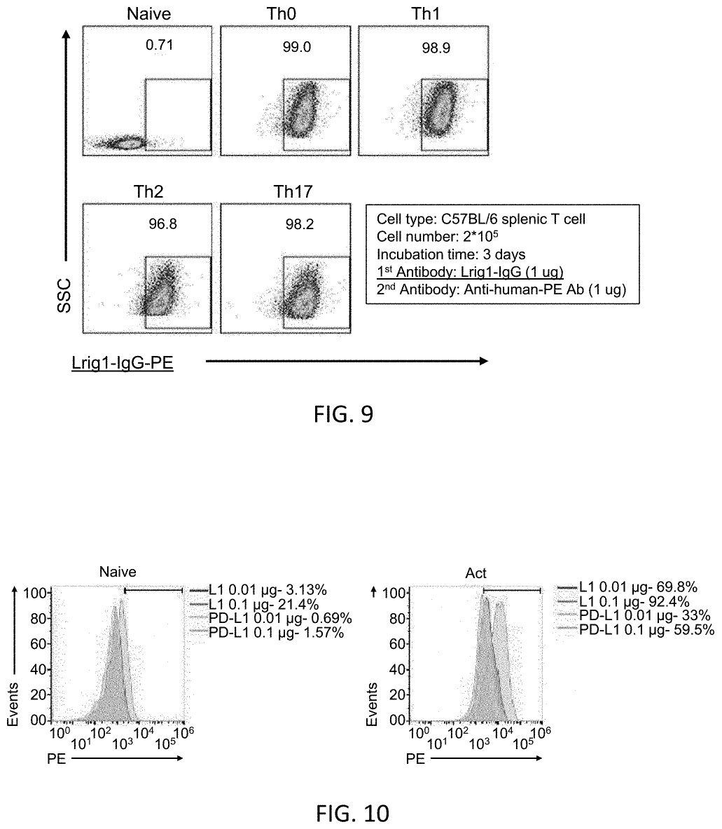

To discover immune cells in which a ligand for Lrig-1 protein is present, naive T cells, thought to be targets of regulatory T cells, were induced to differentiate into activated T cells, Th1 cells, Th2 cells, or Th17 cells, and then these differentiated cells were stained with the fusion protein (primary antibody) of Preparation Example 5 and anti-human-PE antibody (secondary antibody). Levels of the fusion protein of Preparation Example 5 bound to the respective cell surfaces was measured using a fluorescence-activated cell sorter (FACS). The results are illustrated in FIG. 9.

As illustrated in FIG. 9, the fusion protein of Preparation Example 5 hardly binds to the antigen on the surface of naive T cells, and thus weak staining was observed (0.71%); and the fusion protein of Preparation Example 5 binds to the antigen on the surface of activated T cells, Th1 cells, Th2 cells, and Th17 cells at a very high level, and thus staining of 96% or higher was observed.

From these results, it was found that the ligand for the Lrig-1 protein is a surface protein induced by stimulation with a T cell receptor.

### [Example 6] Identification of Expression Level of Lrig-1 Protein in Activated T Cells

For comparison, the surface of naive T cells and activated T cells was stained in a concentration-dependent manner with a PD-L1-IgG fusion protein (product name: rmB7-H1/Fc Chimera, manufactured by R&D Systems, Cat No.: 1019-B7) (hereinafter referred to as ‘PD-L1-IgG’) that can recognize PD-1, which is previously known to be highly expressed in activated T cells and subsets of T cells, or the fusion protein of Preparation Example 5; and levels of the fusion protein of Preparation Example 5 bound to the respective cell surfaces were measured using a cell sorter. The results are illustrated in FIG. 10.

As illustrated in FIG. 10, it was identified that in a case of being stained with the fusion protein of Preparation Example 5, a remarkably high expression level of the fusion protein was observed, in a concentration-dependent manner, on the surface of activated T cells, as compared with the proportion observed in a case of being stained with PD-L1-IgG.

From these results, it was found that the ligand for the Lrig-1 protein was expressed at a high expression level on the surface of activated T cells or subsets of T cells, as compared with naive T cells.

### [Example 7] Identification of Ligand for Lrig-1 Protein in Activated T Cells

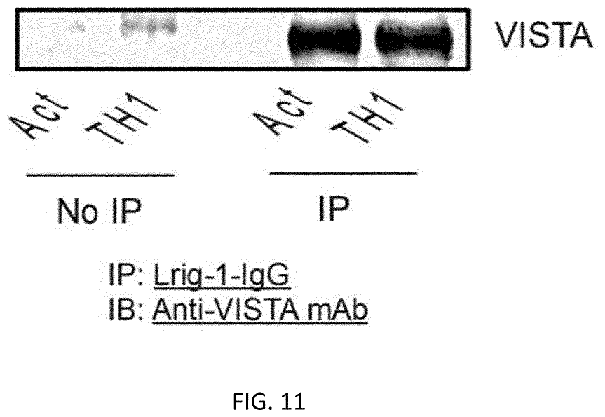

In Example 6, the fact that the ligand for Lrig-1 protein is highly expressed on the surface of activated T cells was identified. Based on this fact, to identify which molecule the ligand expressed on the activated T cells is, cell lysates of activated T cells and Th1 cells were subjected to immunoprecipitation with the fusion protein of Preparation Example 5. Identification was made using various antibodies among candidate ligands for Lrig-1 protein. As a result, Western blot was performed through a monoclonal antibody of V-domain Ig suppressor of T cell activation (VISTA), which is an immune checkpoint. The results are illustrated in FIG. 11.

As illustrated in FIG. 11, it was found that the molecule bound to the fusion protein of Preparation Example 5 was VISTA. In addition, it was found that VISTA was bound to the fusion protein of Preparation Example 5 at a very high level in both activated T cells (Act) and Th1 cells (TH1). From these results, it can be seen that the ligand for the Lrig-1 protein is VISTA.

### [Example 8] Identification of Interaction Between VISTA in Activated T Cells and Lrig-1 Protein

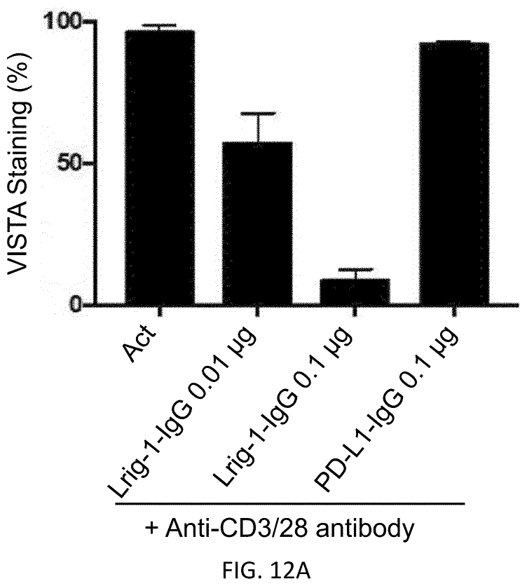

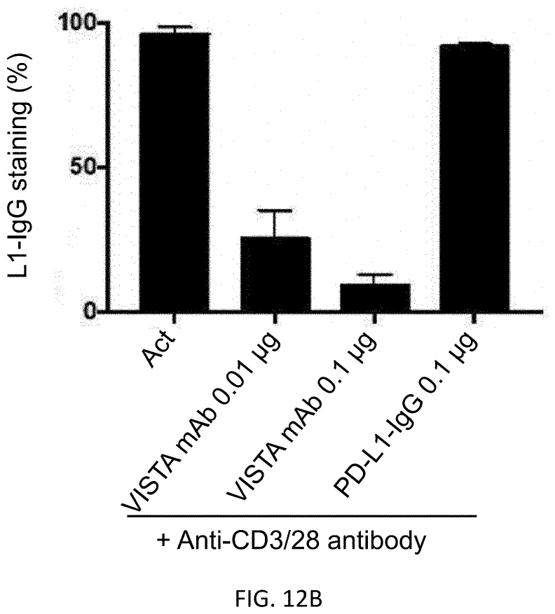

To identify whether VISTA existing on the surface of activated T cells directly interacts with Lrig-1 protein, VISTA mAb was bound to the surface of activated T cells in a concentration-dependent manner, and then the activated T cells were subjected to staining with the fusion protein of Preparation Example 5. Then, a degree of staining was checked using a fluorescence-activated cell sorter (FACS). The results are illustrated in FIGS. 12A and 12B.

As illustrated in FIG. 12A, it was found that in a case where the cell surface was stained only with the fusion protein of Preparation Example 5, the fusion protein exhibited a binding rate of about 95%, whereas in a case where the VISTA mAb was bound in a concentration-dependent manner before staining with the fusion protein of Preparation Example 5, the fusion protein exhibited a remarkably decreased binding rate due to competition.

On the contrary, as illustrated in FIG. 12B, it was found that even in a case where the fusion protein of Preparation Example 5 was first bound to the surface of activated T cells and then the VISTA mAb was bound thereto, a rate at which VISTA was stained decreased.

From these results, it was found that VISTA is expressed on the surface of the activated T cells, and the Lrig-1 protein recognizes and interacts with VISTA.

### [Example 9] Identification of Expression Pattern of VISTA on Surface of T Cells

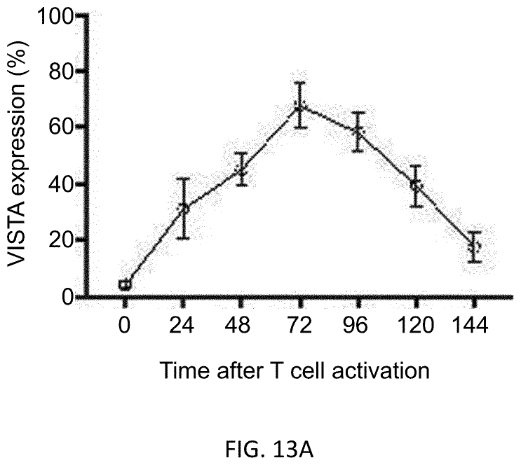

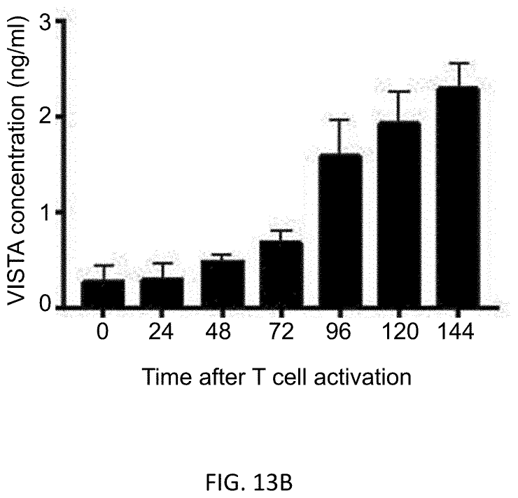

To identify an expression pattern of VISTA on the surface of T cells, mouse naive T cells were activated with an anti-CD3/28 antibody, and then expression levels of VISTA were checked at respective time points. The results are illustrated in FIG. 13A.

As illustrated in FIG. 13A, it was found that the expression level of VISTA gradually increased until 72 hours of activation; however, after 96 hours had elapsed, the expression level thereof on the surface gradually decreased.

To identify the cause of the above-mentioned phenomenon, expression levels of an extracellular domain of VISTA present in a cell culture medium of activated T cells were measured at respective time points through ELISA that recognizes the extracellular domain of VISTA. The results are illustrated in FIG. 13B.

As illustrated in FIG. 13B, it was found that the extracellular domain of VISTA was hardly detected in the culture medium until 72 hours had elapsed after activation; however, the expression level of the extracellular domain of VISTA rapidly increased in the culture medium after 96 hours had elapsed (FIG. 13B). That is, from the viewpoint that the expression level of the extracellular domain of VISTA shows a pattern directly opposite to the expression level of VISTA present on the surface of activated T cells, it was found that the extracellular domain of VISTA in the culture medium is secreted by cleavage of VISTA present on the surface of the activated T cells.

### [Example 10] Identification of Correlation Between Secretion of Extracellular Domain of VISTA and Onset of Autoimmune Disease

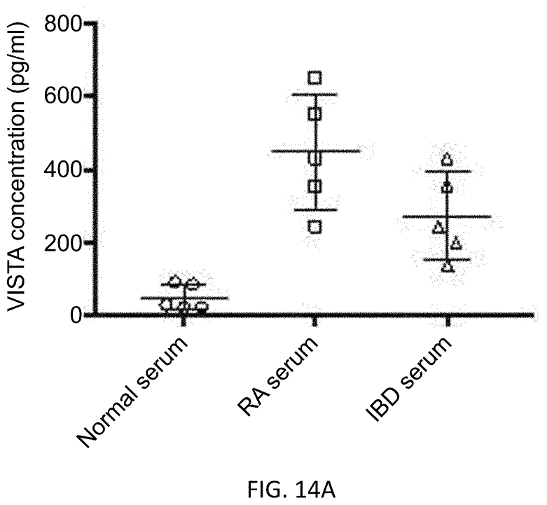

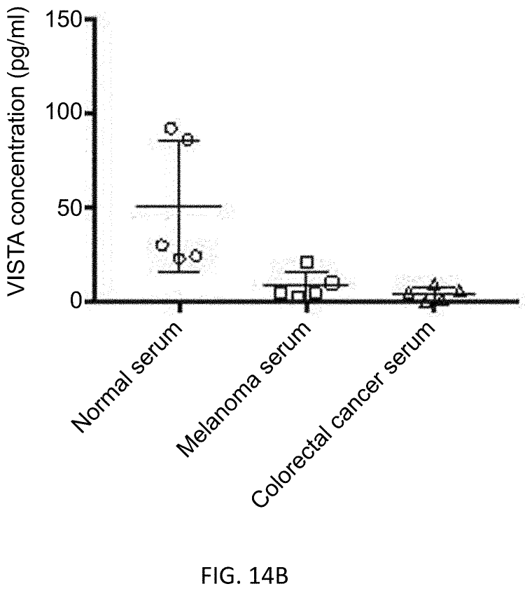

To investigate correlation between secretion of an extracellular domain of VISTA and onset of an autoimmune disease, serum was prepared from a mouse animal model, in which an autoimmune disease of rheumatoid arthritis (RA) or inflammatory bowel disease (IBD) was induced, and then an amount of an extracellular domain of VISTA present in the serum was analyzed by ELISA using a VISTA-specific antibody. The results are illustrated in FIGS. 14A and 14B.

As illustrated in FIGS. 14A and 14B, it was found that the extracellular domain of VISTA was detected in a larger amount in the serum of the mice, in which the autoimmune disease of RA or IBD was induced, than in the serum of normal mice.

### [Example 11] Identification of Correlation Between Secretion of Extracellular Domain of VISTA and Onset of Autoimmune Disease

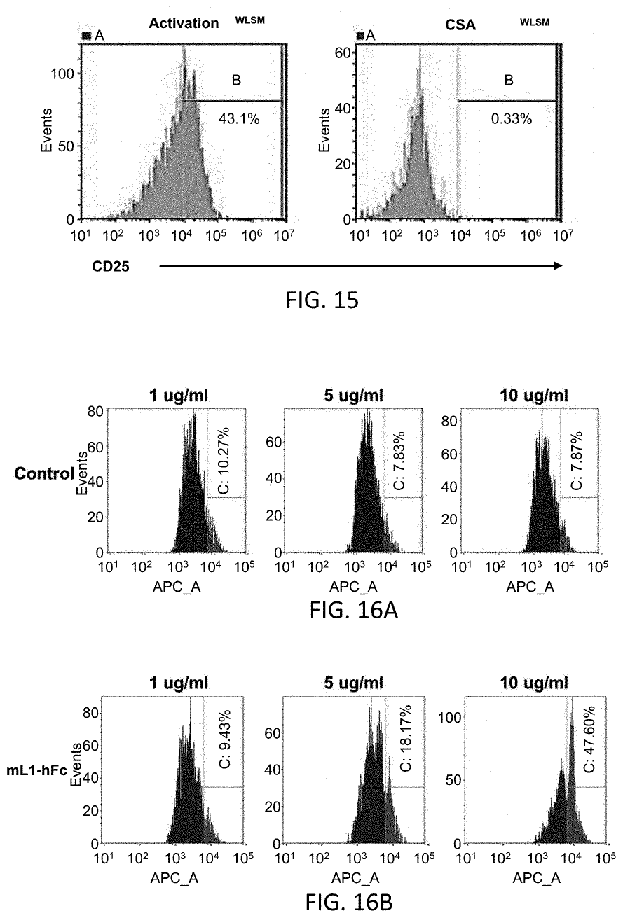

CD4+CD62L+ T cells, which are naive T cells, were isolated from the spleen of 8-week-old C57BL/6 mice, and then the naive T cells were activated using an anti-CD3/anti-CD28 mAb immobilized on a plate. Thereafter, a degree of activation of the T cells was determined by analyzing, with FACS, an expression level of CD25 surface protein, which is expressed by induction on the surface of the T cells. The results are illustrated in FIG. 15. As illustrated in FIG. 15, for a control with suppressed activation of T cells, in a case where T cells were treated with cyclosporine (CsA), which is an immunosuppressant used in clinical practice, it can be seen that activation of the T cells was inhibited, resulting in decreased expression of CD25 from 43.1% to 0.33%.

From these results, it can be seen that in a case where VISTA was stimulated with Lrig1-IgG, which is a ligand for VISTA, a co-inhibitory signal was induced in activated T cells, thereby remarkably inhibiting activation of T cells, and the fusion protein of Preparation Example 5 exhibited a much better degree of inhibition of T cell activation than PD-L1-IgG that stimulates PD-1.

Furthermore, it can be seen that in a case where the fusion protein of Preparation Example 5 is administered in combination with the extracellular domain of VISTA or a fragment thereof; or a fusion protein that includes the extracellular domain of VISTA or a fragment thereof and immunoglobulin, a co-inhibition signal can be more efficiently induced in T cells, thereby more effectively inducing activation of the T cells.

### [Example 12] Therapeutic Effect of Fusion Protein According to Present Invention on Immune-Related Diseases

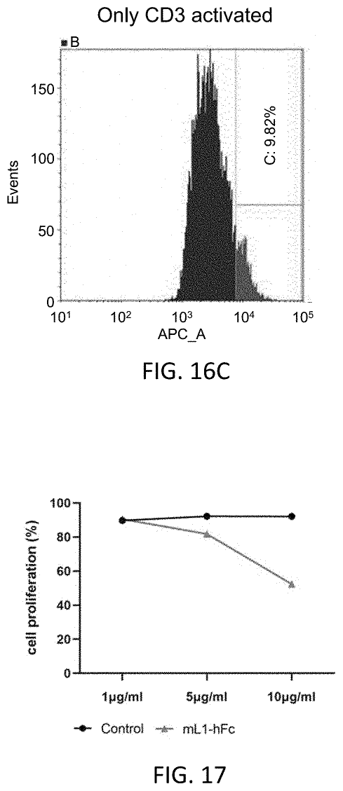

CD4+CD62L+ T cells, which are naive T cells, were isolated from the spleen of 8-week-old C57BL/6 mice, and then the naive T cells were activated using an anti-CD3/anti-CD28 mAb immobilized on a plate. Thereafter, the activated T cells were dispensed into a 96-well plate. Then, the 96-well plate was treated with 1 μg/ml of anti-CD3 mAb and the fusion protein of Preparation Example 5 that corresponds to a concentration of 1 μg/ml, 5 μg/ml, or 10 μg/ml, and then subjected to staining with CSFE. Then, a degree of proliferation of the cells was analyzed under observation with FACS. The results are illustrated in FIGS. 16A, 16B, 16C and 17. Here, for a control, an anti-FLAG antibody was used in place of the fusion protein of Preparation Example 5.

As illustrated in FIGS. 16A, 16B, 16C and 17, in a case of being treated with the control and CD3 alone, the degree of inhibition of proliferation of the activated T cells corresponded to a maximum of 7.87% or 9.82%, whereas in a case of being treated with Lrig-1-IgG (mL1-hFc), which is the fusion protein of Preparation Example 5, proliferation of the activated T cells was inhibited by 18.17% at a concentration of 5 μg/ml and by 47.60% at a concentration of 10 μg/ml (FIGS. 17 and 18).

From these results, it can be seen that in a case where activated T cells are treated with the fusion protein according to the present invention, a co-inhibitory signal can be induced in the activated T cells, thereby remarkably inhibiting activation of the T cells. Thus, the fusion protein according to the present invention can be applied very effectively to prevention, amelioration, or treatment of immune-related diseases caused by activated T cells.

Although the present invention has been described in detail above, the scope of the present invention is not limited thereto. It will be obvious to those skilled in the art that various modifications and changes can be made without departing from the technical spirit of the present invention described in the claims.

### INDUSTRIAL APPLICABILITY

The fusion protein provided in the present invention interacts with a ligand for Lrig-1 protein, which is present in activated T cells, to give a co-inhibitory signal to the activated T cells, so that death or suppressed activity of the activated T cells is induced, which allows the fusion protein to be very effectively used in the field of prevention, amelioration, or treatment of immune-related diseases.

1-22. (canceled)

23. A method for preventing or treating an immune-related disease, the method comprising administering, to an individual, a fusion protein that includes an extracellular domain of leucine-rich and immunoglobulin-like domains-1 (Lrig-1) protein and an immunoglobulin Fc region; a nucleic acid molecule encoding the fusion protein; an expression vector containing the nucleic acid molecule; or a host cell transformed with the expression vector.

24. The method according to claim 23, wherein the extracellular domain of the Lrig-1 protein consists of the amino acid sequence represented by SEQ ID NO: 1 or 3.

25. The method according to claim 23, wherein the immunoglobulin Fc region includes 1 to 4 domains selected from the group consisting of CH1, CH2, CH3, and CH4 domains.

26. The method according to claim 23, wherein the immunoglobulin Fc region is an IgG-, IgA-, IgD-, IgE-, or IgM-derived Fc region, heavy chain constant region 2 (CH2), heavy chain constant region 3 (CH3), hinge, a fragment thereof, or a combination thereof, or a hybrid Fc including the combination.

27. The method according to claim 23, wherein the immunoglobulin Fc region includes IgG-, IgA-, IgD-, IgE-, or IgM-derived CH2 and CH3 domains.

28. The method according to claim 23, wherein the immunoglobulin Fc region includes IgG1-, IgG2-, IgG3-, or IgG4-derived CH2 and CH3 domains.

29. The method according to claim 23, wherein the immunoglobulin Fc region includes a hinge region, the hinge region including an IgG-, IgA-, IgM-, IgD-, IgE-, or Abatacept-derived hinge region.

30. The method according to claim 29, wherein the IgG-, IgA-, IgM-, IgD-, IgE-, or Abatacept-derived hinge region includes an IgG1-, IgG2-, IgG3-, IgG4-, IgD-, or Abatacept-derived hinge region.

31. The method according to claim 23, wherein the immunoglobulin Fc region includes CH2 and CH3 which are represented by SEQ ID NO: 5 or 6.

32. The method according to claim 23, wherein the immunoglobulin Fc region includes a hinge region represented by any one of SEQ ID NOs: 7 to 10.

33. The method according to claim 23, wherein the extracellular domain of the Lrig-1 protein is directly linked to the N-terminus or C-terminus of the immunoglobulin Fc region.

34. The method according to claim 23, wherein the extracellular domain of the Lrig-1 protein is linked, via a linker, to the N-terminus or C-terminus of the immunoglobulin Fc region.

35. The method according to claim 34, wherein the linker is at least one of a peptide linker represented by SEQ ID NO: 11 and a peptide linker represented by SEQ ID NO: 12.

36. The method according to claim 34, wherein the linker is at least one selected from the group consisting of peptide linkers, each of which consists of an amino acid sequence represented by any one of Formulas 1 to 4:
(G)ₙ [Formula 1]
(GGGGS)ₘ [Formula 2]
(EAAAK)ₚ [Formula 3]
(XP)_{q} [Formula 4]
wherein
n, m, and p are each independently an integer of 1 or higher;
q is an integer of 5 to 40; and
X is any one amino acid selected from the group consisting of A (Ala), C (Cys), D (Asp), E (Glu), F (Phe), G (Gly), H (His), I (Ile), K (Lys), L (Leu), M (Met), N (Asn), O (Pyl), P (Pro), Q (Gln), R (Arg), S (Ser), T (Thr), U (Sec), V (Val), W (Trp), and Y (Tyr).

37. The method according to claim 34, wherein the linker is at least one selected from the group consisting of peptide linkers represented by SEQ ID NOs: 96 to 100.

38. The method according to claim 23, further comprising:
a step of administering, to an individual,
a V-domain Ig suppressor of T cell activation (VISTA) or a fragment thereof, or
a fusion protein that includes VISTA or a fragment thereof and an immunoglobulin Fc region.

39. The method according to claim 23, wherein the immune-related disease is at least any one selected from the group consisting of autoimmune diseases; graft versus host disease; organ transplant rejection; asthma; atopy; or acute or chronic inflammatory diseases.

40. The method according to claim 39, wherein the autoimmune disease is at least one selected from the group consisting of rheumatoid arthritis, type 1 diabetes, systemic scleroderma, systemic lupus erythematosus, atopic dermatitis, psoriasis, alopecia areata, asthma, Crohn's disease, Behcet's disease, Sjogren's syndrome, Guillain-Barre syndrome, chronic thyroiditis, multiple sclerosis, multiple myositis, ankylosing spondylitis, fibromyalitis, and polyarteritis nodosa.

41. The method according to claim 39, wherein the autoimmune disease is a neurodegenerative disease or neuroinflammatory disease.

42. The method according to claim 41, wherein the neurodegenerative disease or neuroinflammatory disease is at least one selected from the group consisting of stroke, dementia, Alzheimer's disease, Parkinson's disease, Huntington's disease, Niemann-Pick disease, prion disease, Creutzfeldt-Jakob disease, frontal temporal dementia, Lewis dementia, amyotrophic lateral sclerosis, paraneoplastic syndrome, corticobasal degeneration, multisystematic atrophy, progressive nuclear paralysis, autoimmune disease in nervous system, spinocerebellar ataxia, inflammatory and neuropathic pain, cerebrovascular disease, spinal cord injury, and tauopathy.


SEQUENCE LISTING (100 sequences)

SEQ ID NO: 1 - protein, 762 aa, Homo sapiens
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIF

SEQ ID NO: 2 - DNA, 2286 bp, Homo sapiens
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttc

SEQ ID NO: 3 - protein, 760 aa, Mus musculus
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTT

SEQ ID NO: 4 - DNA, 2280 bp, Mus musculus
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccaca

SEQ ID NO: 5 - protein, 206 aa, Homo sapiens
LFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 206 aa, Mus musculus
IFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - protein, 26 aa, Homo sapiens
EPKSSDKTHTSPPCPAPELLGGPSVF

SEQ ID NO: 8 - protein, 27 aa, Mus musculus
EPRGPTIKPCPPCKCPAPNLLGGPSVF

SEQ ID NO: 9 - protein, 39 aa, Homo sapiens
RNTGRGGEEKKKEKEKEEQEERETKTPECPSHTQPLGVF

SEQ ID NO: 10 - protein, 26 aa, Artificial Sequence
  Abatacept hinge
EPKSSDKTHTSPPSPAPELLGGSSVF

SEQ ID NO: 11 - protein, 2 aa, Artificial Sequence
  peptide linker
GS

SEQ ID NO: 12 - protein, 3 aa, Artificial Sequence
  peptide linker
GGG

SEQ ID NO: 13 - protein, 994 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 995 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 1007 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 994 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 2982 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcgagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 18 - DNA, 2985 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcgagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 19 - DNA, 3021 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttccgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 20 - DNA, 2982 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcgaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 21 - protein, 992 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 993 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 1005 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 992 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 2976 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 26 - DNA, 2979 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 27 - DNA, 3015 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacacgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 28 - DNA, 2976 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 29 - protein, 992 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPKSSDKTHTSPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 30 - protein, 993 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 1005 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTRNTGRGGEEKKKEKEKEEQEERETKTPECPSHTQPLGVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 32 - protein, 992 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTEPKSSDKTHTSPPSPAPELLGGSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - DNA, 2976 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagagccaaagtcctctgataagacacacacctctccaccatgcccagcaccagagctgctgggaggaccaagcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 34 - DNA, 2979 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagagcctcggggccctaccatcaagccctgccccccttgcaagtgccctgcccctaatctgctgggcggaccctccgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 35 - DNA, 3015 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacacgcaacaccggccgcggcggcgaggagaagaagaaggagaaggagaaggaggagcaggaggagcgcgagaccaagacccccgagtgccccagccacacccagcccctgggcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 36 - DNA, 2976 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacagaaccgaaatcttctgacaaaacccacacctctccgccgtctccggctccggaactgctgggtggttcttctgttttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 37 - protein, 996 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 997 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGSEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 1009 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGSRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 996 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGSEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 2988 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggatccgagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 42 - DNA, 2991 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggatccgagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 43 - DNA, 3027 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggatcccgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 44 - DNA, 2988 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggatccgaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 45 - protein, 994 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 995 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 1007 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 994 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 2982 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 50 - DNA, 2985 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 51 - DNA, 3021 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatcccgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 52 - DNA, 2982 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 53 - protein, 994 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPKSSDKTHTSPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - protein, 995 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 55 - protein, 1007 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSRNTGRGGEEKKKEKEKEEQEERETKTPECPSHTQPLGVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 56 - protein, 994 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGSEPKSSDKTHTSPPSPAPELLGGSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - DNA, 2982 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgagccaaagtcctctgataagacacacacctctccaccatgcccagcaccagagctgctgggaggaccaagcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 58 - DNA, 2985 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgagcctcggggccctaccatcaagccctgccccccttgcaagtgccctgcccctaatctgctgggcggaccctccgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 59 - DNA, 3021 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatcccgcaacaccggccgcggcggcgaggagaagaagaaggagaaggagaaggaggagcaggaggagcgcgagaccaagacccccgagtgccccagccacacccagcccctgggcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 60 - DNA, 2982 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggatccgaaccgaaatcttctgacaaaacccacacctctccgccgtctccggctccggaactgctgggtggttcttctgttttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 61 - protein, 999 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGGGGSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 1000 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGGGGSEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 1012 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGGGGSRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 999 aa, Artificial Sequence
  fusion protein
AGPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVGIFGGGGSEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 2997 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggcggtggcggatccgagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 66 - DNA, 3000 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggcggtggcggatccgagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 67 - DNA, 3036 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggcggtggcggatcccgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 68 - DNA, 2997 bp, Artificial Sequence
  fusion protein
gctgggcctcgggctccttgtgctgccgcctgcacatgtgcaggcgattccctggactgcggcggcagaggcctggccgccctgcctggcgatctgccatcctggacccggagcctgaacctgagctacaacaagctgagcgagatcgatcccgccggctttgaggacctgcctaacctgcaggaggtgtatctgaacaataacgagctgaccgcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcggcggtggcggatccgaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 69 - protein, 997 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 998 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPRGPTIKPCPPCKCPAPNLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 1010 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSRNTGRGGEEKKKEKEKEEQEERETKTPECPSHTQPLGVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 997 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 2991 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgagccaaagtcctctgataagacacacacctctccaccatgcccagcaccagagctgctgggaggaccaagcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 74 - DNA, 2994 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgagcctcggggccctaccatcaagccctgccccccttgcaagtgccctgcccctaatctgctgggcggaccctccgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 75 - DNA, 3030 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatcccgcaacaccggccgcggcggcgaggagaagaagaaggagaaggagaaggaggagcaggaggagcgcgagaccaagacccccgagtgccccagccacacccagcccctgggcgtgttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 76 - DNA, 2991 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgaaccgaaatcttctgacaaaacccacacctctccgccgtctccggctccggaactgctgggtggttcttctgttttcctgtttcctccaaagcccaaggacacactgatgatctccaggacaccagaggtgacctgcgtggtggtggacgtgagccacgaggaccccgaggtgaagttcaactggtacgtggatggcgtggaggtgcacaatgccaagaccaagcccagagaggagcagtacaactctacctatagggtggtgagcgtgctgacagtgctgcaccaggactggctgaacggcaaggagtataagtgcaaggtgagcaataaggccctgcctgccccaatcgagaagacaatctccaaggccaagggccagccaagagagccccaggtgtacaccctgccccctagcagggatgagctgacaaagaaccaggtgtccctgacctgtctggtgaagggcttttatccctccgacatcgccgtggagtgggagtctaatggccagcctgagaataactacaagacaaccccacccgtgctggattctgacggcagcttctttctgtattctaagctgaccgtggacaagagcaggtggcagcagggcaacgtgttcagctgctccgtgatgcacgaagcactgcacaatcactacacccagaaatcactgtcactgagccctggcaaa

SEQ ID NO: 77 - protein, 997 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPKSSDKTHTSPPCPAPELLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 78 - protein, 998 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 79 - protein, 1010 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSRNTGRGGEEKKKEKEKEEQEERETKTPECPSHTQPLGVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 80 - protein, 997 aa, Artificial Sequence
  fusion protein
AQAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTGGGGSEPKSSDKTHTSPPSPAPELLGGSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - DNA, 2991 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgagccaaagtcctctgataagacacacacctctccaccatgcccagcaccagagctgctgggaggaccaagcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 82 - DNA, 2994 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgagcctcggggccctaccatcaagccctgccccccttgcaagtgccctgcccctaatctgctgggcggaccctccgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 83 - DNA, 3030 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatcccgcaacaccggccgcggcggcgaggagaagaagaaggagaaggagaaggaggagcaggaggagcgcgagaccaagacccccgagtgccccagccacacccagcccctgggcgtgttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 84 - DNA, 2991 bp, Artificial Sequence
  fusion protein
gctcaggctggacctagggctccttgcgctgccgcctgcacctgtgcaggcgattctctggactgcagcggccggggcctggccacactgcccagggacctgccttcctggaccagatctctgaacctgagctacaatcggctgtccgagatcgattctgccgcctttgaggacctgacaaatctgcaggaggtgtatctgaacagcaatgagctgaccgcaatcccctccctgggagcagcctctatcggcgtggtgagcctgttcctgcagcacaacaagatcctgagcgtggatggctcccagctgaagagctacctgtctctggaggtgctggacctgagctccaacaatatcaccgagatcagatctagctgttttcctaatggcctgcggatcagagagctgaacctggcctctaatcggatcagcatcctggagtccggcgccttcgatggcctgagcagatccctgctgacactgcgcctgtccaagaaccggatcacccagctgcccgtgaaggcctttaagctgcctaggctgacacagctggacctgaaccggaatagaatcaggctgatcgagggcctgaccttccagggcctggatagcctggaggtgctgcgcctgcagcggaacaatatctcccgcctgacagacggagcattttggggcctgtctaagatgcacgtgctgcacctggagtacaatagcctggtggaggtgaactctggcagcctgtatggcctgaccgccctgcaccagctgcacctgtccaacaatagcatcagcagaatccagagggatggctggtccttctgccagaagctgcacgagctgatcctgtcttttaacaatctgaccaggctggacgaggagagcctggcagagctgtcctctctgtccatcctgcgcctgtctcacaatgccatcagccacatcgccgagggcgcctttaagggcctgaagagcctgagggtgctggatctggaccacaacgagatctctggcaccatcgaggatacaagcggcgccttcacaggcctggacaatctgtccaagctgaccctgtttggcaacaagatcaagtctgtggccaagcgggccttctctggcctggagagcctggagcacctgaacctgggcgagaatgccatcagatccgtgcagttcgatgcctttgccaagatgaagaatctgaaggagctgtacatcagctccgagagcttcctgtgcgactgtcagctgaagtggctgccaccttggctgatgggaaggatgctgcaggcctttgtgaccgccacatgcgcccacccagagagcctgaagggccagagcatcttctccgtgctgcccgatagcttcgtgtgcgacgattttcctaagccacagatcatcacccagccagagacaacaatggccgtggtgggcaaggacatccggtttacatgttccgccgcctctagctcctctagccccatgaccttcgcctggaagaaggataacgaggtgctggccaatgccgacatggagaacttcgcccacgtgagagcccaggatggcgaagtgatggagtataccacaatcctgcacctgcggcacgtgacctttggccacgagggcagataccagtgcatcatcacaaatcacttcggctctacctatagccacaaggccaggctgacagtgaacgtgctgcctagctttaccaagatcccacacgacatcgccatcagaacaggcaccacagcaaggctggagtgtgcagcaaccggacacccaaaccctcagatcgcatggcagaaggatggaggcacagacttccctgcagcccgcgagaggagaatgcacgtgatgccagacgatgacgtgttctttatcacagatgtgaagatcgatgacatgggcgtgtactcctgcaccgcacagaacagcgccggcagcgtgtccgccaacgccaccctgaccgtgctggagacaccatccctggccgtgcccctggaggacagggtggtgaccgtgggcgagacagtggcctttcagtgtaaggccaccggctctccaacaccaaggatcacctggctgaagggcggcaggcccctgagcctgacagagcgccaccacttcacccctggcaatcagctgctggtggtgcagaacgtgatgatcgatgacgccggcaggtatacatgcgagatgagcaatcctctgggcaccgagagggcacactcccagctgtctatcctgcctaccccaggctgccggaaggatggcaccacaggcggtggcggatccgaaccgaaatcttctgacaaaacccacacctctccgccgtctccggctccggaactgctgggtggttcttctgttttcatcttcccacccaagatcaaggacgtgctgatgatctccctgtcccccatcgtgacctgcgtggtggtggacgtgtccgaggacgaccccgacgtgcagatcagttggttcgtgaacaacgtggaagtgcacaccgcccagacccagacccacagagaggactacaactccaccctgcgggtggtgtccgccctgcccatccagcaccaggactggatgtccggcaaagaattcaagtgcaaagtgaacaacaaggacctgcctgcccccatcgagcggaccatctccaagcccaagggctccgtgcgggctccccaggtgtacgtgctgccccctccagaggaagagatgaccaagaagcaggtcacactgacctgcatggtcaccgacttcatgcccgaggacatctacgtggaatggaccaacaatggcaagaccgagctgaactacaagaacaccgagcctgtgctggactccgacggctcctacttcatgtactccaagctgcgggtggaaaagaagaactgggtcgagcggaactcctactcctgctccgtggtgcacgagggcctgcacaaccaccacaccaccaagtccttctcccggacccccggcaaa

SEQ ID NO: 85 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 86 - protein, 309 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSSNTQGIENPGFETTPPFQGMPEAKTRPPLSYVAQRQPSESGRYLLSDPSTPLSPPGPGDVFFPSLDPVPDSPNSEAI

SEQ ID NO: 87 - DNA, 4714 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatgagctgtcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgccctgggtgcgagaacatggcgcctccagggggcgggaggagcactaggggctggggcaggaggctcctggagcgctggattcctggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 88 - DNA, 4795 bp, Mus musculus
gagcattcactctagcgagcgagcggcgtgtacagccggctccctgggctcctggagtcccgcttgctccaagcgcactccagcagtctctttctgctcttgcccggctcgacggcgacatgggtgtccccgcggtcccagaggccagcagcccgcgctggggaaccctgctccttgctattttcctggctgcatccagaggtctggtagcagccttcaaggtcaccactccatattctctctatgtgtgtcccgagggacagaatgccaccctcacctgcaggattctgggccccgtgtccaaagggcacgatgtgaccatctacaagacgtggtacctcagctcacgaggcgaggtccagatgtgcaaagaacaccggcccatacgcaacttcacattgcagcaccttcagcaccacggaagccacctgaaagccaacgccagccatgaccagccccagaagcatgggctagagctagcttctgaccaccacggtaacttctctatcaccctgcgcaatgtgaccccaagggacagcggcctctactgctgtctagtgatagaattaaaaaaccaccacccagaacaacggttctacgggtccatggagctacaggtacaggcaggcaaaggctcggggtccacatgcatggcgtctaatgagcaggacagtgacagcatcacggctgcggccctggccaccggcgcctgcatcgtgggaatcctctgcctcccccttatcctgctgctggtctataagcagagacaggtggcctctcaccgccgtgcccaggagttggtgaggatggacagcagcaacacccaaggaatcgaaaacccaggcttcgagaccactccacccttccaggggatgcctgaggccaagaccaggccgccactgtcctatgtggcccagcggcaaccttcggagtcaggacggtacctgctctctgaccccagcacacctctgtcgcctccaggccctggggacgtctttttcccatccctagatccagtccctgactcccctaactctgaagccatctaaaccagctggggaaccatgaaccatggtacctgggtcagggatatgtgcacttgatctatggctggcccttggacagtcttttaggcactgactccagcttccttgctcctgctctgagcctagactctgcttttacaagatgcacagaccctcccctatctctttcagacgctacttggggggcagggagaagatgttggattgctcatggctgttctcaagatcttgggatgctgagttctccctagagacttgacttcgacagccacagatgtcagatgacctgcatcctatgaacgtccggcttggcaagagcctttcttcatggaaaccagtagcccggaggggatgaggtaggcaccttgccaccctcccgggagagagacacaagatgtgagagactcctgctcactgtgggggtgtggctggcctgcttgtttgcctgaggatgctcctctgttggactgactctatccccctggattctggagcttggctggcctatgtcccaccagaggagcatctcagcagccttccaccagcaacctgagggcctgccagcttcgtggctctgggctctcattacctgtatggccgtccacagagctcagtggccagaggctttgaaacaggaagtacatgtcaggttcaggaaccactgtgagctcattagtgtcttgagcaatgtgaggcctggaccagtggacacggagggagggtggcgagaggatgatggggatgatgaggggaacacgctcccttcctgtccttgtcatccaccactaccactattcagtgtggagcagtggcaaaggtgaccgacctccacaatgtcctagtgatgctggaccatttctaagtgtgaaagagatgctattaaaaacagtatgtggcaatggctgccaacagctgagtggactggaggcactggctttaaggccctggaggtgcagggcccggtatggggatagggatgggagtttcagtgagggcctagggatcactccgcttctgaccactcttcttctgagcctcacctcagggtgaccttcaggcacacagaagagcttgcccctggtccgatactactcttggctctcatctccagggtttggcatgacctgggcacacagggggagtcttcagaaaggattttaaagcatgaaaagaaagggtagttcttgtgaggtagggatgggcagctgatgtttgagagtgaggagggatacggctgggcagatcactctccagtctctagagggaaagtagctctaagtctgggagagcagcagcccagtggtaccatatgtcttcttgcagcttccactggctgggctgaactgggcatgggtaggaaagctcctgttctgggcctgcagccagggagaaccccattcattccctgaggacagatgggtggggagagaagagagagtttcaggccgggaagcagcaataagctatctgctggggacccagacaagttgtctgatgaggtccaagatgtgggatgccagttatacctggggcttggggatccttagaggctttgtatcatcatcataggagtgtcggggtggccagggcatcaaagccatgacccctgttttatcctcagggtccactcttctgcaccatccattgctctagatctatgcagttactatagacagaatgtgttgttctgtttggctttggggataatggcctggcgaactgccagctgttcagtggcagggctgtgaggccagtcaaagactagaacccacagaccagctgaacgatgagtatagcctgtcccctgggggagcctgacctgtctccagccctaagcttcagacctcaccactcagatgacttctaagaatttgcctgtggggacccctgcatggctgcagctccgtggaaaggagaggaggcccccagcagaagaaccactcgcttcctgcccagcttcctcctgtagggctctaagtctcttcttcttgggaccctgcaagcaaaggcatgtcagcttggtggtttcctgttttgggtgaagttttgtgtggtccgggttctgtctacatccatgaacttggggtgctaccaccttgctgctgctgtagagacagctgcaggatcttagggtggaaaatggaggtgccctgaggtgctagcccttggggcaaaagatggggtggcaatgagacacagtggggaactgagttccccaagaggagggaggagccctgtagcctcaagggccatattgggttcctggtaccagcaaaagcctagagagcgaagtctgtattttgaggaggtaattgatccttacggaatccatcagaaatttggagcgggtgctttatctatctctggagggtctctacctatctccgatgaagctctccctgggcctgggatgggagaaaccaggaggaaaggtgtctgataaagcaggggcttcttgacaagccaaagggccactggtagctgttgtggaccgagctgaccctgctgaagtattgtagtgtgccttggaccaacttctcaaaagagcaaccccggggctaccctacttctgccaggaagaggcggagaaggggctgagaggcctggaaggggctagctccttctttgagaactgctccccggaggacttggaggaggcggctaggctacgggctgctgagggccctttgtctttcctaacctgggcactgttaggatgctccctcctggaaaaggctttcctgggtgtgagctagagcagtgtccatgccagcgctgaacctgccatggtgggagctgaactaaaaatttctcagggaactaaaataggcaaaagaggaactgggggaggagggtgccaggcaggatggggggaagggagggcagtgcaaaagtctcttgaaacacagacagcccagctgagtgccagtcccagatcacagagaatacggctcatctggctcatgttctgcatgcttgctgctttaccctggcactttccttctccaccatgagtgcgagtcctgggagtcctgggagggtgaggattaatgccagcctggggagcagatagctgacagagtccttgggtaactggcttgaaccaggacctcaggattccactctggggatctagctttgtctgggccagtgaagatctctataatggcattattgccaggggataaacatttcactgggttctgatctgttgggtgtggcttcctggaaaatatggtgagaggaattctgctaaggatacagttgataagaaagttctgagattgattagtaatgcctgccttggactcaggaagggaagtggcagtatgaatgccatgtcttaatcattttggttaaaatatgcttcccaaaagatttccacgtgtgttcttgtttatttgacatctgtctccatatcagtcttgaaagcctttctgtgtgtatatatatgatgtttgcgtgtatatatgtttttgtgtgtgcatatggaagtcagaaatcactgggtgtcttcctccattcctttgcaatgtatgttttttttttttttacgatttatttactatatgaatgttttgcctgaatacatgcataggtgtcacgtacatgcctgctggaacgcttggaactggagttacaggtggctatgagctacagtgtgagcactgggaatcaaacctgggtcttctgcaagagcaacaaattaaaagtcagctcttaactacttgagctatttttccaactcc

SEQ ID NO: 89 - protein, 162 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 90 - protein, 159 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAA

SEQ ID NO: 91 - protein, 95 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHG

SEQ ID NO: 92 - protein, 158 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSEN

SEQ ID NO: 93 - protein, 63 aa, Homo sapiens
NFSITMRNLTLLDSGLYCCLVVEIRHHHSEHRVHGAMELQVQTGKDAPSNCVVYPSSSQDSEN

SEQ ID NO: 94 - protein, 94 aa, Mus musculus
FKVTTPYSLYVCPEGQNATLTCRILGPVSKGHDVTIYKTWYLSSRGEVQMCKEHRPIRNFTLQHLQHHGSHLKANASHDQPQKHGLELASDHHG

SEQ ID NO: 95 - protein, 65 aa, Mus musculus
NFSITLRNVTPRDSGLYCCLVIELKNHHPEQRFYGSMELQVQAGKGSGSTCMASNEQDSDSITAA

SEQ ID NO: 96 - protein, 46 aa, Artificial Sequence
  peptide linker
AEAAAKEAAAKEAAAKEAAAKALEAEAAAKEAAAKEAAAKEAAAKA

SEQ ID NO: 97 - protein, 5 aa, Artificial Sequence
  peptide linker
PAPAP

SEQ ID NO: 98 - protein, 12 aa, Artificial Sequence
  peptide linker
AEAAAKEAAAKA

SEQ ID NO: 99 - protein, 6 aa, Artificial Sequence
  peptide linker
IEGRMD

SEQ ID NO: 100 - protein, 7 aa, Artificial Sequence
  peptide linker
DIEGRMD